(19) United States
(12) Patent Application Publication
(10) Pub. No.: US 2018/0317415 A1
(43) Pub. Date: Nov. 8, 2018

(54) NANOBIONIC LIGHT EMITTING PLANTS

(71) Applicant: MASSACHUSETTS INSTITUTE OF TECHNOLOGY, Cambridge, MA (US)

(72) Inventors: Michael S. STRANO, Lexington, MA (US); Seongyeon KWAK, Cambridge, MA (US); Juan Pablo Giraldo GOMEZ, Claremont, CA (US); Min Hao WONG, Cambridge, MA (US)

(73) Assignee: MASSACHUSETTS INSTITUTE OF TECHNOLOGY, Cambridge, MA (US)

(21) Appl. No.: 15/773,166

(22) PCT Filed: Nov. 4, 2016
(86) PCT No.: PCT/US16/60704

§ 371 Date: May 2, 2018

Related U.S. Application Data
(60) Provisional application No. 62/251,071, filed on Nov. 4, 2015.

(51) Int. Cl. A01H 3/00; C12N 9/02; A01G 7/00

(52) U.S. Cl. CPC A01H 3/00; A01G 7/00; C12Y 113/12007; C12N 9/0069

(57) ABSTRACT

A plant nanobionic approach can utilize a system of four nanoparticle types, including luciferase conjugated silica, luciferin releasing poly(lactic-co-glycolic acid), coenzyme A functionalized chitosan, and semiconductor nanocrystal phosphors for wavelength modulation.

### CLAIM OF PRIORITY

This application claims the benefit of prior U.S. Provisional Application No. 62/251,071 filed on Nov. 4, 2015, which is incorporated by reference in its entirety.

### FEDERAL SPONSORSHIP

This invention was made with Government support under Grant No. DE-FG02-08ER46488 awarded by the U.S. Department of Energy. The Government has certain rights in the invention

### FIELD OF INVENTION

This invention relates to nanobionic engineering of photosynthetic organisms.

### BACKGROUND

As independent energy sources, plants are adapted for persistence and self-repair in harsh environments with negative carbon footprints. See Giraldo, J. P. et al. Plant nanobionics approach to augment photosynthesis and biochemical sensing. Nat Mater 13, 400-408, doi:10.1038/nmat3890 (2014), which is incorporated by reference in its entirety.

A eukaryotic cell is a cell that contains membrane-bound organelles, most notably a nucleus. An organelle is a specialized subunit within a cell that has a specific function, and can be separately enclosed within its own lipid bilayer. Examples of organelles include mitochondria, chloroplasts, Golgi apparatus, endoplasmic reticulum, and as previously mentioned, the nucleus. Organelles are found within the cell cytoplasm, an intracellular fluid that is separated from extracellular fluid by the plasma membrane. The plasma membrane is a double layer (i.e., a bilayer) of phospholipids that permits only certain substances to move in and out of the cell.

In addition to these features, plant cells include specialized organelles that are not generally found in animal cells. For example, plant cells include a rigid cell wall. Plant cells also include chloroplasts. Chloroplasts are chlorophyll-containing double-membrane bound organelles that perform photosynthesis. Chloroplasts are believed to be descendants of prokaryotic cells (e.g., cyanobacteria) that were engulfed by a eukaryotic cell.

### SUMMARY OF THE INVENTION

A method of delivering a composition into a plant can include submerging the plant in an chamber, wherein the chamber contains water and the composition; and applying an external pressure to the chamber, thereby generating an inward flow through stomata pores of a plant leaf and infiltrating the composition into the plant.

The method can further include localizing the composition in an organelle, a cell, or a tissue of the plant. The organelle can be selected from the group consisting of a nucleus, endoplasmic reticulum, Golgi apparatus, chloroplast, chromoplast, gerontoplast, leucoplast, lysosome, peroxisome, glyoxysome, endosome and vacuole. The cell can be a stomata guard cell. The tissue can be mesophyll.

The external pressure can be no less than 1.8 bar. A water contact angle on a surface of the plant can be less than 113°. The external pressure can be applied at a velocity less than 0.4 bar/s. The composition can include particles having a size of less than 20 nm, or less than 10 nm. The composition can include a nanoparticle.

A light emitting compound can be immobilized on the nanoparticle. The light emitting compound can be luciferase. The nanoparticle can include a nanotube. The nanoparticle can include a carbon nanotube. The nanoparticle can include a single-walled carbon nanotube. The nanoparticle can include a polymer. The polymer can include a polynucleotide. The polynucleotide can include poly(AT). The polymer includes a polysaccharide. The olysaccharide can be selected from the group consisting of dextran, pectin, hyaluronic acid, chitosan, and hydroxyethylcellulose. The polymer can include poly(ethylene glycol). The nanoparticle can be photoluminescent. The nanoparticle can emit near-infrared radiation. The nanoparticle can be photoluminescent and the photoluminescence emission of the photoluminescent nanoparticle can be altered by a change in a stimulus within the plant. The stimulus can be a concentration of an analyte. The analyte can be a reactive oxygen species. The analyte can be nitric oxide, carbon dioxide, adenosine triphosphate, nicotinamide adenine dinucleotide phosphate, oxygen, or a hazardous gas, such as methane. The stimulus can be a pH of an organelle of the plant. The nanoparticle can be a semiconductor.

The composition can include a dye. The composition can include an enzyme. The composition can include a nutrient. The composition can include a gene.

A green plant can include a composition including a nanoparticle, a silane conjugated with the nanoparticle, and a dye conjugated with the nanoparticle. The silane can be (3-glycidyloxypropyl)trimethoxysilane. The nanoparticles can include silica.

A green plant can include a composition including a nanoparticle, a polymer conjugated with the nanoparticle, and a light-emitting compound immobilized on the nanoparticle via the polymer. The polymer can include poly(ethylene glycol). The light-emitting compound can be luciferase.

A green plant can include a composition including a nanoparticle encapsulated by a light-emitting compound. The light-emitting compound can be luciferin.

A green plant can include a composition including a plurality of nanoparticles and a polysaccharide conjugated with each of the plurality of nanoparticles, wherein a chemical compound is encapsulated by the plurality of nanoparticles. The polysaccharide can be chitosan. The chemical compound can be coenzyme A.

A green plant can include a composition including a nanoparticle, a polymer conjugated with the nanoparticle, and a enzyme immobilized on the nanoparticle via the polymer. The polymer can include poly(ethylene glycol). The enzyme can be luciferase.

Other aspects, embodiments, and features will be apparent from the following description, the drawings, and the claims

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1H show nanoparticles for light emitting plant and light production in vitro.

FIG. 1A-1 shows reaction mechanism of light production by firefly luciferase using nanoparticles. FIG. 1A-2 shows simplified structure of nanoparticles to study localization of nanoparticles (SNP-AF) and to create light emitting plants (SNP-Luc, PLGA-LH and CS-CoA). FIG. 1B shows schematic illustration of infusion and localization of nanoparticles in plant tissues. FIG. 1C shows micrographs of nanoparticles. Transmission electron microscopy (TEM) image of SNP-Luc (left), and scanning electron microscopy (SEM) images of CS-CoA (middle) and PLGA-LH2 (right). Releasing kinetics of PLGA-LH₂ (FIG. 1D) and CS-CoA nanoparticles (FIG. 1E) for 24 h at room temperature. FIG. 1F shows comparison of light production between with and without nanoparticles. FIG. 1G shows comparison of light duration between high and low concentration of SNP-Luc at high concentration of CS-CoA with limited PLGA-LH₂. FIG. 1H shows light duration at different concentration of SNP-Luc at a high concentration of PLGA-LH₂ and CS-CoA.

FIGS. 2A-2F show pressurized bath infusion of nanoparticles (PBIN). FIG. 2A shows a whole watercress plant in a pressurized chamber. FIG. 2B shows contact angle of water drop on both leaf adaxial and abaxial sides of watercress, arugula, spinach and kale leaves. FIG. 2C shows correlation between the net inward acceleration and water drop contact angle of the leaves. FIG. 2D shows applied pressure at different speeds, (1)-(5) spinach leaves, and (6) whole watercress plant (3.5 week-old). FIG. 2E-1 shows shows fluorescence confocal micrographs of dye labeled silica nanoparticles and PLGA nanoparticles in plant tissues. SNP-AF or BODIPY®FL (green), cell membrane (FM 464, red), and chloroplast (blue). FIG. 2E-2 shows light emission from both adaxial and abaxial sides of a leaf after PBIN in 3 week-old watercress. FIG. 2F shows optical image of 3-week old kale plant (top) and a light emitting kale after treatment of surfactant (bottom).

FIGS. 3A-3E show decay kinetics of light emission in living watercress plant. FIG. 3A shows effect of incubation time of SNP-Luc on light intensity and duration. FIG. 3B shows photon number decay of light emitting plants. FIG. 3C shows time-lapsed photos of light emitting plant with PLGA-LH and SNP-Luc. FIG. 3D shows time-lapsed photos of light emitting plant with CS-CoA, PLGA-LH, and SNP-Luc (scale bar 1 cm). FIG. 3E shows the relation between maximum number of photons and light duration in living plants.

FIGS. 4A-4I show wild type light emitting plants. FIG. 4A shows illuminating MIT logo printed on the leaf of arugula (left) and spinach (right). FIG. 4B shows turning on and off of the light emitting plant. FIG. 4C shows illumination of a book with light emitting plants. FIG. 4D shows schematic illustration of shifting the emission wavelength between QD and luciferase-luciferin reaction by resonance energy transfer. FIG. 4E shows shifted nIR emission spectrum in a cuvette (left) and in a watercress leaf (right) obtained by spectrofluorometer with no laser excitation at 0.1 s integration time. FIG. 4F shows diagrammatic depiction of set-up with Raspberry Pi with Night vision camera masked with a long pass filter to monitor nIR emission. FIG. 4G shows shifted emission from the living watercress (3 week-old), brightfield (left), and false-colored image of nIR emission in Image J (right). FIG. 4H shows nIR signal as a response to the external chemical, luciferin (recolored). FIG. 41 shows design of a syringe applicator with the letter ‘M’ drawn in AutoCad (left) and 3D printed syringe applicators generated by Lulzbot mini (right).

FIGS. 5A-5B show fluorescence confocal micrographs of spinach leaves infiltrated by LIN. Leaf discs of spinach plants 3 week-olds were infiltrated with SNP-AF488 (green) via LIN and cell membranes were stained with FM 464 fluorescent dye (red). FIG. 5A shows confocal images of leaf region in which the nanoparticles where infiltrated with a needleless syringe. FIG. 5B shows near the infiltrated region. Leaf epidermal cells (0 μm depth, left panel) and leaf mesophyll cells (5 μm depth, right).

FIG. 6 shows fluorescence confocal micrographs of the adaxial side of spinach leaves infiltrated with SNP7-AF (green) via PBIN. Cell membranes labeled with FM-464 (red), and chloroplast fluorescence emission (blue). Leaf epidermal cells (0 μm depth, left panel) and leaf mesophyll cells (5 μm depth, right). Leaf discs were taken from the plants 2 h after infiltration.

FIGS. 7A-7C show PBIN method applied to kale plants. FIG. 7A shows whole kale plant inside glass syringe containing nanoparticle buffered solution. FIG. 7B shows comparison of differential pressure measured during PBIN with water. FIG. 7C shows the adaxial and abaxial side of kale leaf after PBIN and drying leaf surface.

FIG. 8 shows fluorescence confocal micrographs of spinach leaves infiltrated by PBIN at 0.4 bar/s.

FIGS. 9A-9C show fluorescence confocal micrographs of spinach leaves at different incubation times of nanoparticles within the plant. FIG. 9A shows leaf cut and discs prepared immediately after LIN. FIG. 9B shows leaf cut immediately after LIN and leaf discs prepared 2 h later. FIG. 9C shows infiltrated leaf was kept in the dark and attached to living plant for 2 h, then leaf discs were prepared for imaging. Leaf epidermal cells (0 μm depth, left panel) and leaf mesophyll cells (5 μm depth—middle and 10 μm—right pannel).

FIGS. 10A-10B show effect of incubation time of SNP-Luc on luminescence in living plant. FIG. 10A shows whole watercress plant having even glowing patches after PBIN without pre-incubation of SNP-Luc. FIG. 10B shows fluorescence confocal micrographs of spinach at 0 h incubation (left) and 2 h incubation (right) of SNP-AF.

FIGS. 11A-11D show in vitro decay kinetics of nanoparticle complex luminescence. FIG. 11A shows luminescence intensity at different concentrations of ATP with luciferin and SNP-Luc. FIG. 11B shows luminescence intensity of the initial and 3 mins later at different concentration of ATP with luciferin and SNP-Luc. FIG. 11C shows luminescence intensity at different concentration of luciferin, with ATP and SNP-Luc. FIG. 11D shows luminescence intensity at different SNP-Luc, with ATP and luciferin.

FIG. 12 shows control experiments for nIR emission from the QD-Luc infiltrated plant. Images of watercress leaf infiltrated with SNP-Luc, luciferin and ATP.

FIG. 13 shows schematic illustration of kinetic model of light production by firefly luciferase, luciferin and coenzyme A in the presence of ATP.

FIG. 14 shows the model plot (black line), which accounted for the reaction rates and releasing kinetics of nanoparticles showed great fit with experimental data (red line).

### DETAILED DESCRIPTION

The engineering of plants for light emission has been proposed by genetic modification for sustainable illumination, since plants possess independent energy sources, negative carbon footprints, and autonomous self-repair. See, Ow, D. W. et al. Transient and stable expression of the firefly luciferase gene in plant cells and transgenic plants. Science 234, 856-859, doi:10.1126/science.234.4778.856 (1986), Krichevsky, A., Meyers, B., Vainstein, A., Maliga, P. & Citovsky, V. Autoluminescent Plants. Plos One 5 (11), doi:ARTN e15461. dot:10.1371/journal.pone.0015461 (2010), and Giraldo, J. P. et al. Plant nanobionics approach to augment photosynthesis and biochemical sensing. Nat Mater 13, 400-408, doi:10.1038/nmat3890 (2014), each of which is incorporated by reference in its entirety. Plants are therefore compelling platforms for engineering new functions, such as light emission and information transfer. Attempts to generate luminescent plants have focused on genetic engineering using either the firefly luciferase gene or bacterial lux operon. See Ow, D. W. et al. Transient and stable expression of the firefly luciferase gene in plant cells and transgenic plants. Science 234, 856-859, doi:10.1126/science.234.4778.856 (1986), and Krichevsky, A., Meyers, B., Vainstein, A., Maliga, P. & Citovsky, V. Autoluminescent Plants. Plos One 5 (11), doi:ARTN e15461. dot:10.1371/journal.pone.0015461 (2010), each of which is incorporated by reference in its entirety. A central complication with this approach is difficulty in localizing enzymatic and luciferin producing regions with those of high ATP concentration, requiring external administration of 1 mM luciferin in the case of the former, a reagent with poor aqueous solubility and toxic to the plant above 400 μM. Recent advances in the engineering of nanoparticles able to traffic within and localize to specific organelles within living plants offer new opportunities to control the location and concentrations of light generating reactions within the living, wild-type plant. See Giraldo, J. P. et al. Plant nanobionics approach to augment photosynthesis and biochemical sensing. Nat Mater 13, 400-408, doi:10.1038/nmat3890 (2014), and Boghossian, A. A. et al. Application of Nanoparticle Antioxidants to Enable Hyperstable Chloroplasts for Solar Energy Harvesting. Adv Energy Mater 3, 881-893, doi:10.1002/aenm.201201014 (2013), each of which is incorporated by reference in its entirety.

Described herein is a plant nanobionic approach that utilizes the size and surface charges of four distinct nanoparticle types to control their distribution in and around the plant mesophyll, generating light emitting variants of several common wild-type plants such as spinach (Spinacia oleracea), arugula (Eruca sativa), and watercress (Nasturtium officinale).

A method of delivering a composition into a plant can include submerging the plant in an chamber, wherein the chamber contains water and the composition, and applying an external pressure to the chamber, thereby generating an inward flow through stomata pores of a plant leaf and infiltrating the composition into the plant. The method can further include localizing the composition in an organelle, a cell, or a tissue of the plant. The organelle can be a nucleus, endoplasmic reticulum, Golgi apparatus, chloroplast, chromoplast, gerontoplast, leucoplast, lysosome, peroxisome, glyoxysome, endosome, or vacuole. The cell can be a stomata guard cell. The tissue can be mesophyll.

In this method, the external pressure can be preferably between 0 and 2 bars, but any pressure from 0 to infinite can work for the system.

A water contact angle on a surface of the plant can be preferably between 0 and 113°.

The external pressure can be applied at any velocity between 0 and infinite as long as the infusion into a living plant does not incur damage to the plant, preferably between 0.02 and 0.4 bar/s.

The size of the composition can be less than 20 nm, less than 15 nm, less than 10 nm, or less than 5 nm.

The firefly luciferase-luciferin reaction pathway is a commonly employed system for reacting ATP within an organism to generate yellow-green photoemission, centered at 560 nm, via the oxidation of D-luciferin catalyzed by luciferase in the presence of ATP and Mg²⁺. See, Deluca, M. Firefly luciferase. Adv Enzymol Relat Areas Mot Blot 44, 37-68 (1976), Nakatsu, T. et al. Structural basis for the spectral difference in luciferase bioluminescence. Nature 440, 372-376, doi:10.1038/nature04542 (2006), and Seliger, H. H. & Mc, E. W. Spectral emission and quantum yield of firefly bioluminescence. Arch Biochem Biophys 88, 136-141 (1960), each of which is incorporated by reference inits entirety.

Four kinds of nanoparticle compositions with controlled size and surface charge were synthesized to target each within specific compartments of the leaf.

In one embodiment, a composition can include a nanoparticle, a silane conjugated with the nanoparticle, and a dye conjugated with the nanoparticle. The silane can be (3-glycidyloxypropyl)trimethoxysilane. The nanoparticles can include silica.

In another embodiment, a composition can include a nanoparticle, a polymer conjugated with the nanoparticle, and a light-emitting compound or an enzyme immobilized on the nanoparticle via the polymer. The polymer can include poly(ethylene glycol). The light-emitting compound can be luciferase.

In another embodiment, a composition can include a nanoparticle encapsulated by a light-emitting compound. The light-emitting compound can be luciferin.

In another embodiment, a composition can include a plurality of nanoparticles and a polysaccharide conjugated with each of the plurality of nanoparticles, wherein a chemical compound is encapsulated by the plurality of nanoparticles. The polysaccharide can be chitosan. The chemical compound can be coenzyme A.

A green plant can include a composition including a nanoparticle, a polymer conjugated with the nanoparticle, and a enzyme immobilized on the nanoparticle via the polymer. The polymer can include poly(ethylene glycol). The enzyme can be luciferase.

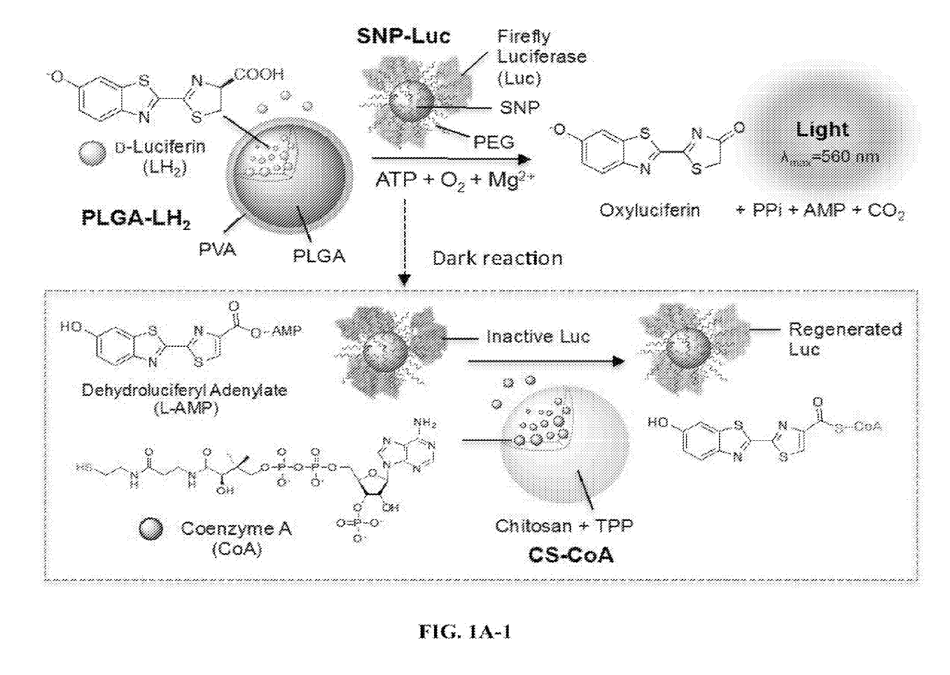

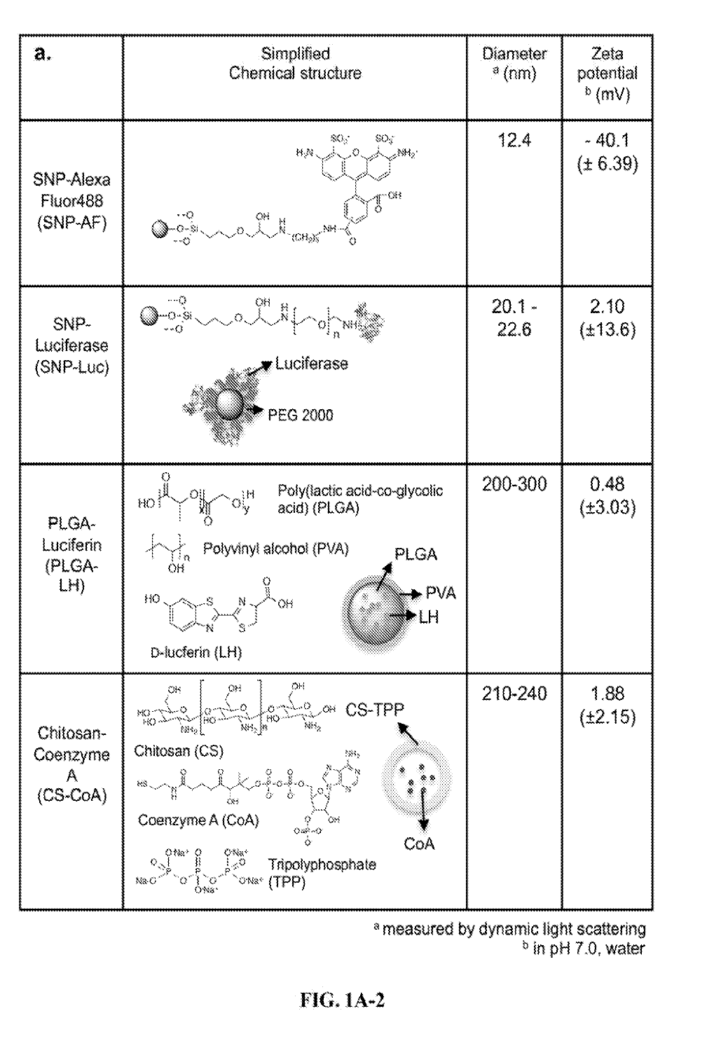

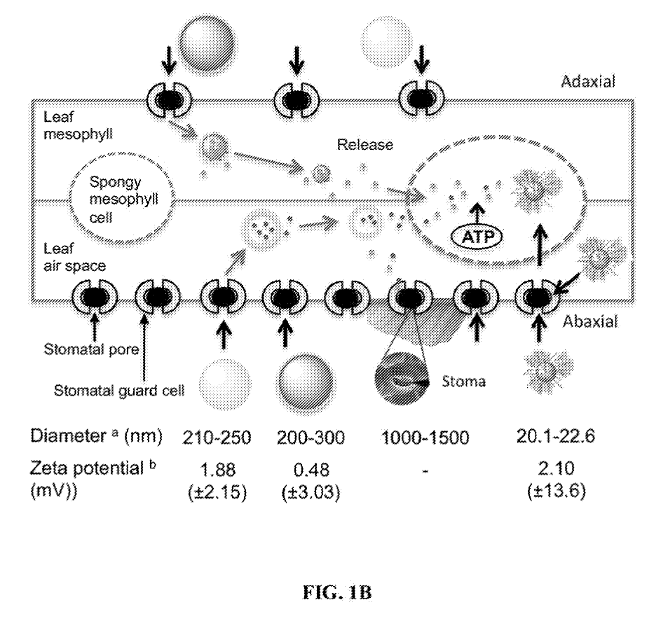

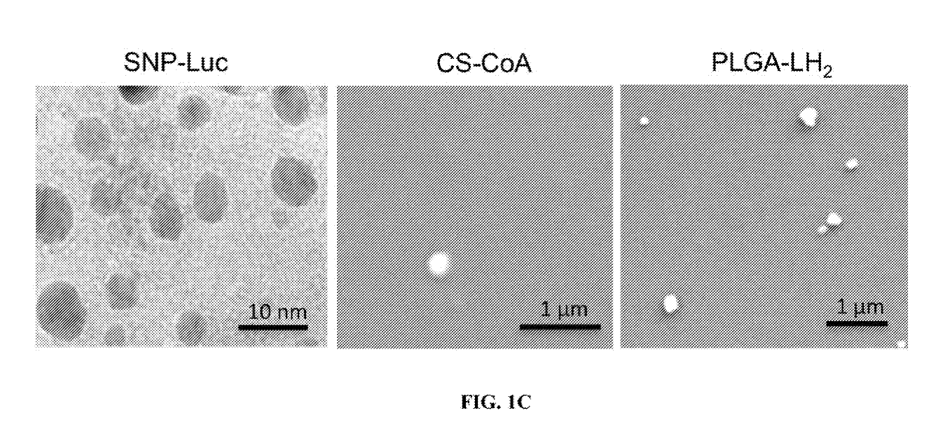

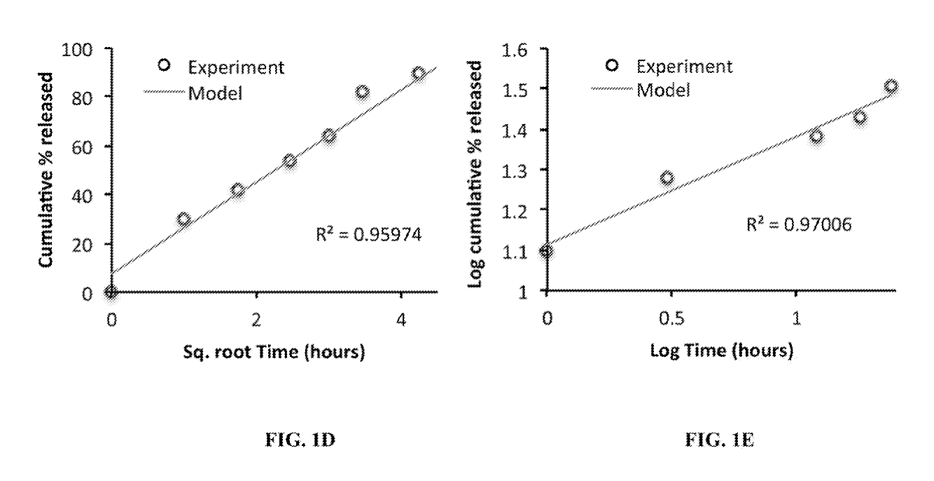

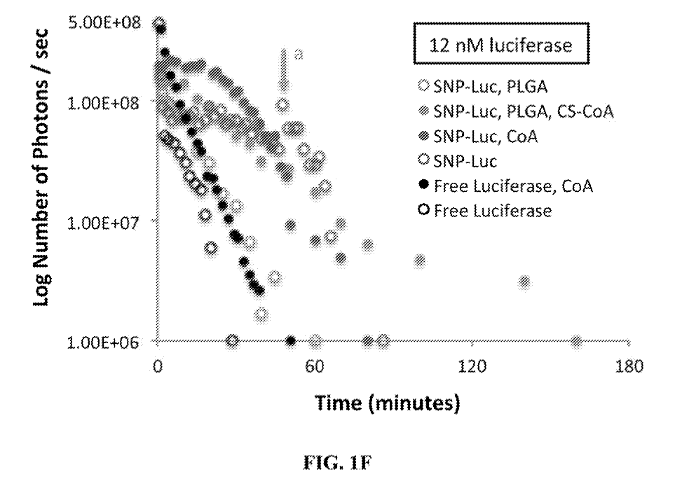

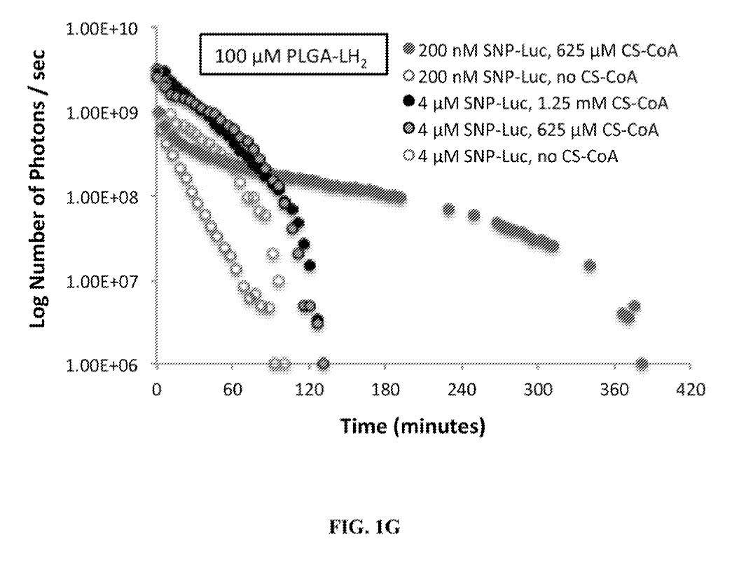

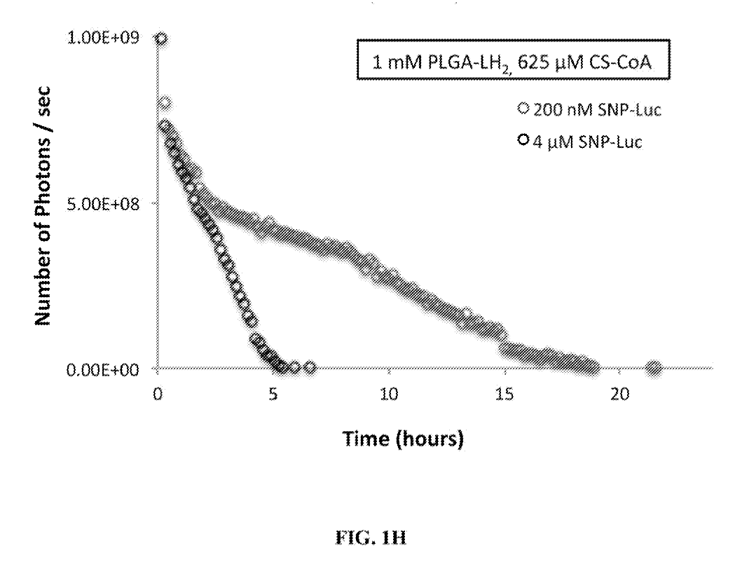

FIG. 1A-1 shows reaction mechanisim of light production by firefly luciferase using nanoparticles. In the presence of adenosine triphosphate (ATP), oxygen (O₂) and magnesium ions (Mg²⁺), the firefly luciferase immobilized silica nanoparticles (SNP-Luc) catalyze the oxidation of luciferin that is released from luciferin-encapsulated PLGA nanoparticles (PLGA-LH₂). Dehydrolucifery-adenylate (L-AMP) is formed as a by-product, acting as a strong inhibitor of the luciferase. Coenzyme A (CoA) released from CoA-encapsulated chitosan nanoparticles (CS-CoA) opposes this inhibitory effect of L-AMP by triggering the thiolytic reaction, which regenerates luciferase activity. FIG. 1B shows that nanopartcles are infiltrated through the stomatal pores on the abaxial and adaxial sides. SNP-Luc (<20 nm) can enter the stomatal guard cells and the mesophyll cells, whereas PLGA-LH2 and CS-CoA (>200 nm) stay in the mesophyll and release luciferin and coenzyme A, respectively. The released chemicals can enter the cytosol, where ATP exists in high concentration. Hydrohynamic diameters and zeta potential of the nanoparticles are shown below the illustration. Here, 7 nm but not 20 nm or larger silica nanoparticles are able to efficiently traverse the plant cell membrane and can be used to localize a high concentration of luciferase conjugated versions within stomata guard cells and leaf mesophyll cells.

To overcome luciferin toxicity in most plants and low aqueous solubility, 300 nm PLGA nanoparticle carriers were synthesized to supply a high extracellular flux of luciferin within the leaf mesophyll intercellular spaces. Coenzyme A extends the light emission by regenerating luciferase activity as reacting with strong inhibitor of light production, dehydroluciferyl-adenylate (IC₅₀=5 nM). See, Fraga, H., Fernandes, D., Fontes, R. & Esteves da Silva, J. C. Coenzyme A affects firefly luciferase luminescence because it acts as a substrate and not as an allosteric effector. Febs J 272, 5206-5216, doi:10.1111/j.1742-4658.2005.04895.x (2005), and Marques, S. M. & Esteves da Silva, J. C. Firefly bioluminescence: a mechanistic approach of luciferase catalyzed reactions. IUBMB Life 61, 6-17, doi:10.1002/iub.134 (2009), each of which is incorporated by reference in its entirety. Furthermore, conjugation of luciferase to a semiconductor nanocrystal or other fluorescent nanoparticle shifts the emission to any alternative wavelength accessible by resonant energy transfer. See, Alam, R. et al. Near infrared bioluminescence resonance energy transfer from firefly luciferase-quantum dot bionanoconjugates. Nanotechnology 25, doi:10.1088/0957-4484/25/49/495606 (2014), Frangioni, J. V. Self-illuminating quantum dots light the way. Nat Biotechnol 24, 326-328, doi:10.1038/nbt0306-326 (2006), and So, M. K., Xu, C., Loening, A. M., Gambhir, S. S. & Rao, J. Self-illuminating quantum dot conjugates for in vivo imaging. Nat Biotechnol 24, 339-343, doi:10.1038/nbt1188 (2006), each of which is incorporated by reference in its entirety.

FIG. 1F shows comparison of light production between with and without nanoparticles, evaluation was carried out in a 1 mL mixture containing 12 nM luciferase, 100 μM luciferin, and 100 μM ATP with or without 100 μM coenzyme A (n=2). SNP-Luc (blue open circles) showed longer light duration than that of free luciferase (black open circles). The addition of CoA extended light duration increased light production (closed circles). Light duration was significantly extended with SNP-Luc, PLGA-LH2 and CS-CoA (red circles). FIG. 1G shows comparison of light duration between high (4 μM) and low (0.2 μM) concentration of SNP-Luc at high concentration of CS-CoA (625 μM, 1 mM) with limited PLGA-LH₂ (100 μM), CS-CoA couldn't elongate light duration in high concentration of SNP-Luc 4 μM (black circles). In lower concentration of SNP-Luc 200 nM, CS-CoA dramatically extended light duration (blue circles). This reaction was luciferin-limited. FIG. 1H shows light duration at different concentration of SNP-Luc at a high concentration of PLGA-LH₂ (1 mM) and CS-CoA (625 μM). Light duration was extended by 6 hr with 4 μM SNP-Luc (black circles), and by 22 hr with 200 nM SNP-Luc (blue circles). The releasing kinetics of PLGA-LH₂ and CS-CoA affect on reaction rate and enzyme activity. The light intensity was analyzed by Image J from the photos taken with Nikon D5300 at a set of 5 s exposure, f/4.5 and ISO 3200.

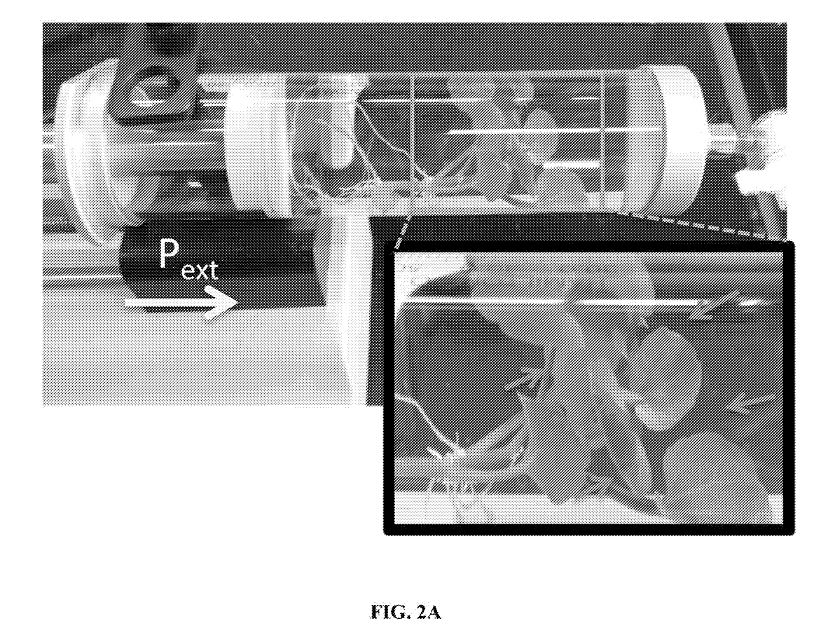

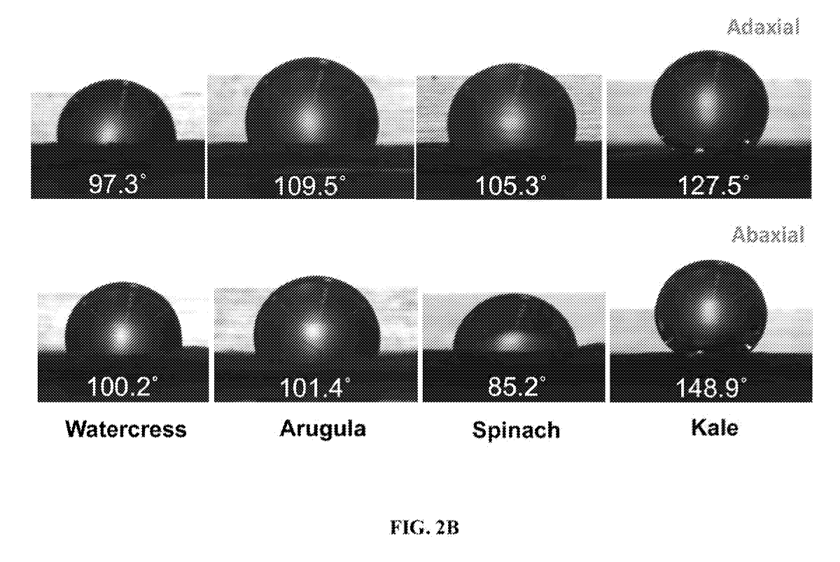

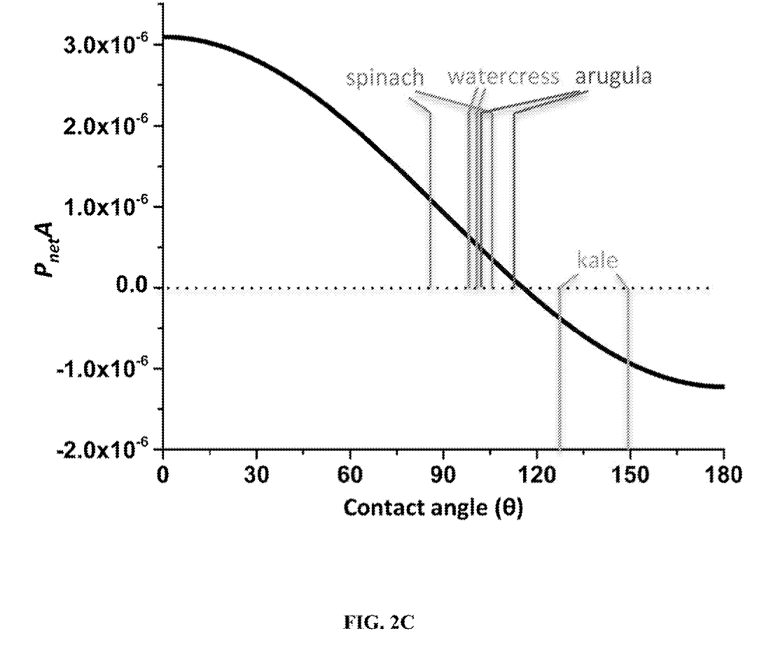

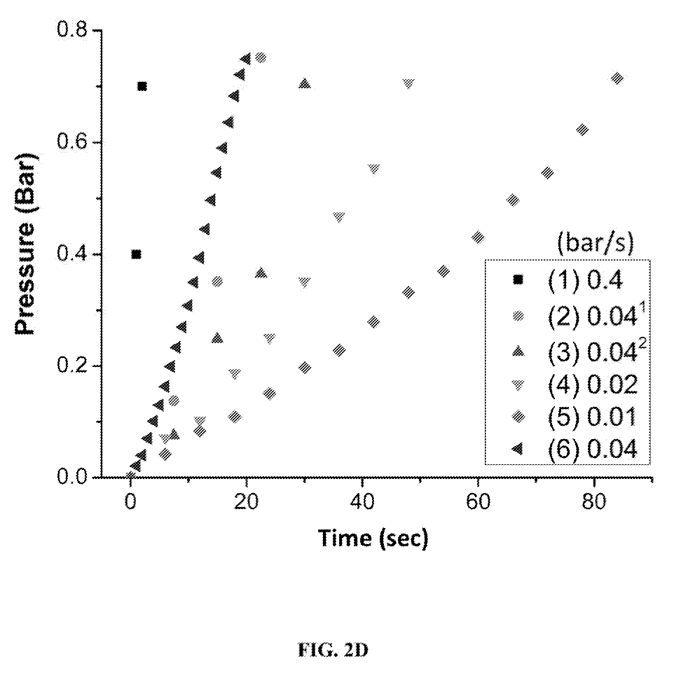

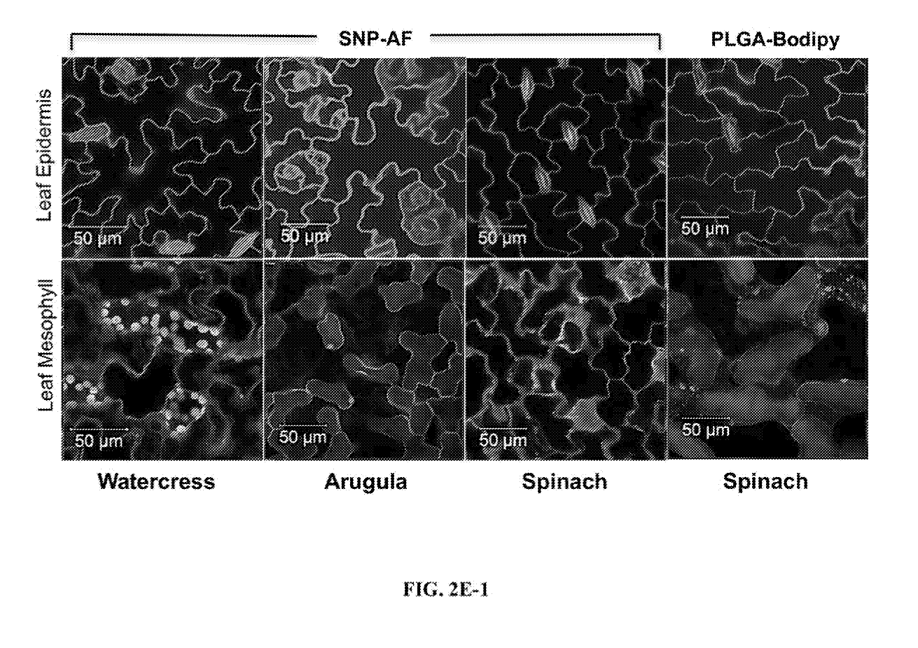

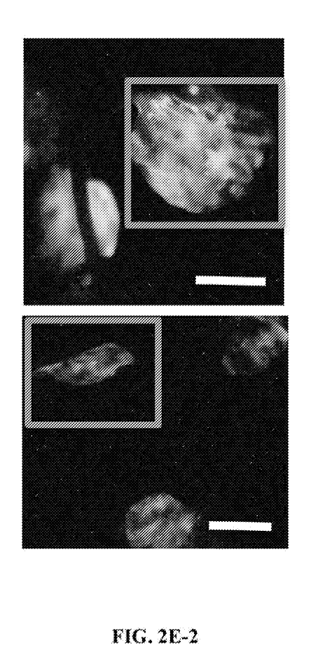

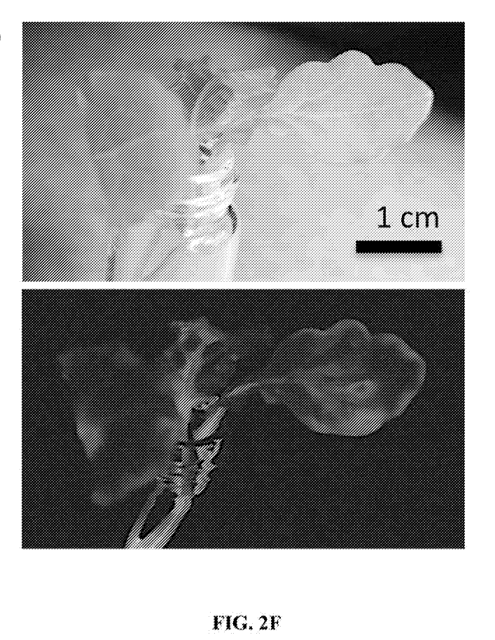

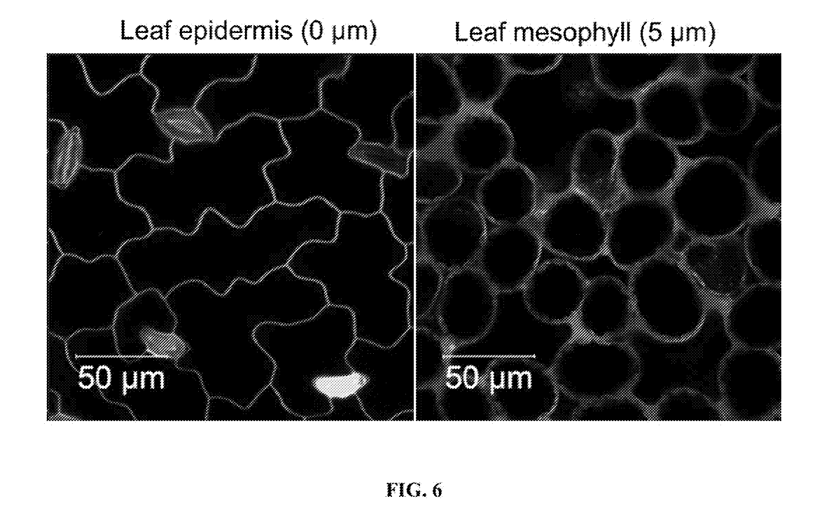

The 10-15 μm stomatal pores on the both adaxial and abaxial sides of a leaf are highly permeable to nanoparticles (FIGS. 2E and 6), but once in the mesophyll, the nanoparticle size and surface charge restrict further localization. See, Giraldo, J. P. et al. Plant nanobionics approach to augment photosynthesis and biochemical sensing. Nat Mater 13, 400-408, doi:10.1038/nmat3890 (2014), and Eichert, T., Kurtz, A., Steiner, U. & Goldbach, H. E. Size exclusion limits and lateral heterogeneity of the stomatal foliar uptake pathway for aqueous solutes and water-suspended nanoparticles. Physiol Plantarum 134, 151-160, doi:10.1111/j.1399-3054.2008.01135.x (2008), each of which is incorporated by reference in its entirety. The luciferase immobilized 7 nm silica nanoparticles (SNP-Luc) can enter leaf mesophyll cells and stomata guard cells, and localize near the organelles, chloroplasts and mitochondria, where ATP generation is highest. The larger, 200-300 nm luciferin encapsulated poly(lactic-co-glycolic acid) (PLGA-LH) and coenzyme A containing chitosan-tripolyphosphate (CS-CoA) nanoparticles, release steady flux of luciferin and coenzyme A within the extracellular space of the leaf mesophyll where they transport through the cell wall and membrane (FIGS. 1A-1B). Uptake and localization are confirmed using fluorescent confocal microscopy analysis. The 7 nm silica nanoparticles labeled with Alexa Fluor 488 (SNP-AF) are observed in every stomata guard cell from all three plant species, spinach, arugula, and watercress, as well as leaf mesophyll cells of watercress and arugula, but not spinach. The stomata open when the guard cells increase in volume, which can happen in minutes and requires rapid and massive transport of solute across the guard cell membrane. See, Schroeder, J. I., Raschke, K. & Neher, E. Voltage Dependence of K+ Channels in Guard-Cell Protoplasts. Proc Natl Acad Sci U S A 84, 4108-4112, doi:DOI 10.1073/pnas.84.12.4108 (1987), which is incorporated by reference in its entirety. The uneven thickness of the stomata guard cell wall and the solute transport through the cell membrane may promote SNP of small enough size to localize within stomata guard cells. See, Evert, R. F. Epidermis. Esaus Pflanzenanatomie: Meristeme, Zellen Und Gewebe Der Pflanzen Ihre Struktur, Funktion Und Entwicklung, 193-232, doi:Book Doi 10.1515/9783110211320 (2009), which is incorporated by reference in its entirety. Alternatively, 200 nm PLGA particles labeled with the dye BODIPY® FL clearly release dye molecules into the intercellular spaces of the mesophyll that then eventually enter the cells (FIG. 2E-1).

Disclosed herein is a method for rapid, whole plant infusion of nanoparticles through the stomata pores using a pressurized bath infusion of nanoparticles (PBIN). Here, the entire plant is briefly submerged in a pressured aqueous chamber to approximately 1.8 bar (FIG. 2A).

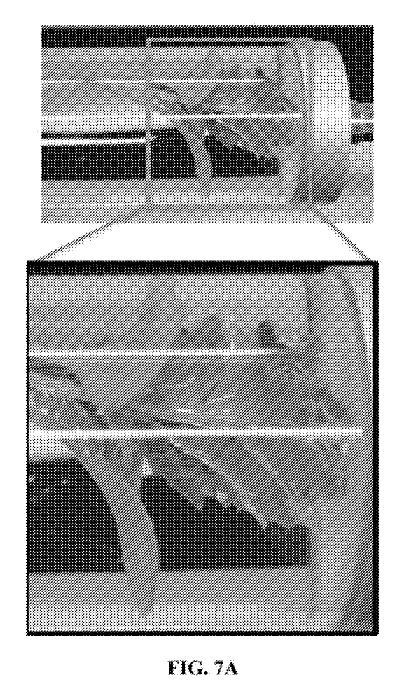

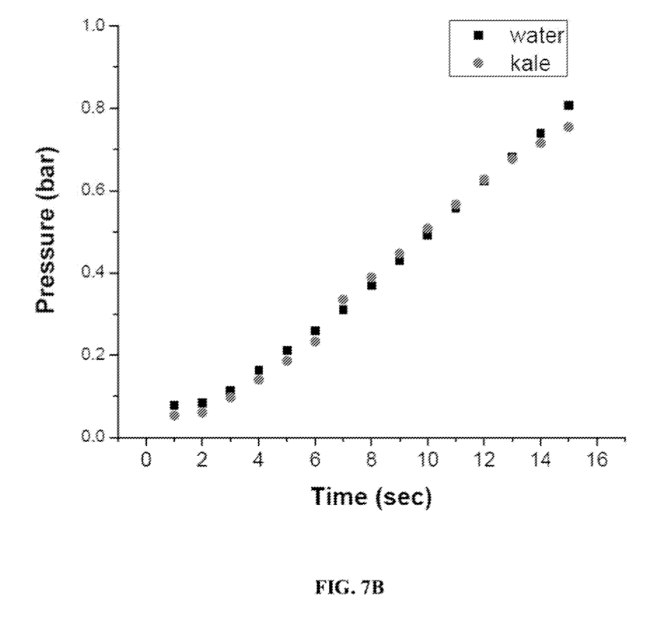

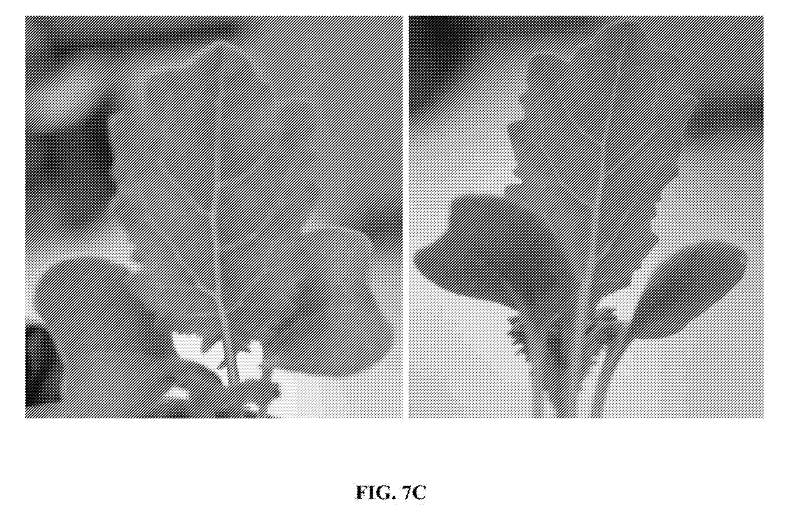

PBIN is able to simultaneously infiltrate the four classes of nanoparticles described above into wild-type spinach, arugula, watercress but not kale (Brassica oleracea) (see FIG. 7). These species were selected because of their empirically observed high ATP production rate. Water contact angle measurement on kale leaves show values of 127.5° at the adaxial side, and 148.9° at the abaxial side, but significantly higher than that of spinach, watercress and arugula, which range from 85.2° to 109.5° (FIG. 2B). PBIN works by supplying an external pressure against the internal microchannels within the mesophyll of the plant, generating an inward flow through the stomata pores. The net inward acceleration is dictated by the sum of the capillary forces, viscous drag, resistance from trapped air compression and the applied PBIN force (Eq. 1). See Phan, V. N. et al. Capillary Filling in NanochannelsModeling, Fabrication, and Experiments. Heat Transfer Eng 32, 624-635, doi:10.1080/01457632.2010.509756 (2011), which is incorporated by reference in its entirety.

$${P}_{nst} A = 2 σ w cos θ - \frac{12 μ \overline{u}}{h} wx - \frac{{hwp}_{0} x}{l - x} + {P}_{ext} A \qquad Eq . 1$$

σ is the surface tension of water at 25° C. (0.07197 J/m²), w is the diameter of open stomatal pore (1.5×10⁻⁷ m), and 0 is the contact angle of water drop on the leaf surface (varying). Here, μ is the dynamic viscosity of water at 25° C. (1.002×10⁼³ Ns/m²), μ̅ is the filling speed determined from PBIN (4.5×10⁻³ m/s), and his the height of the channel, same as w (1.5×10⁻⁷ m). ρ₀ is the initial pressure of the trapped air, i.e. atmospheric pressure (101325 N/m²), the filling length x (1×10⁻² m) and the total length of microchannel/(1.8×10⁻² m) are estimated from thickness of mesophyll, infiltrated length, and length of an entire leaf. Pₑₓₜ is the external applied pressure (135000 N/m²), and A is the cross sectional area of the stomatal pore (1.7×10⁻¹⁰ m²). Interestingly, using these values into Eq. 1 predicts favorable PBIN infiltration if the plant leaf contact angle is less than 113° (FIG. 2C), in perfect agreement with the findings.

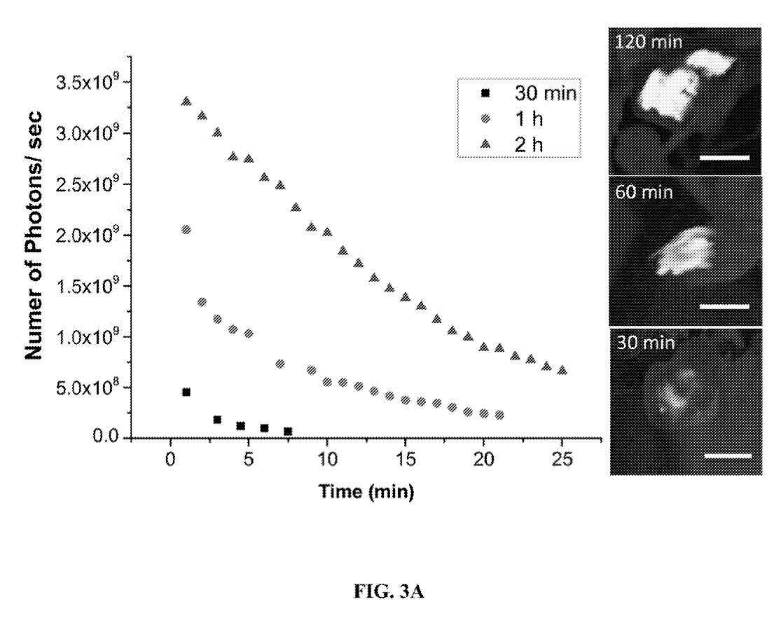

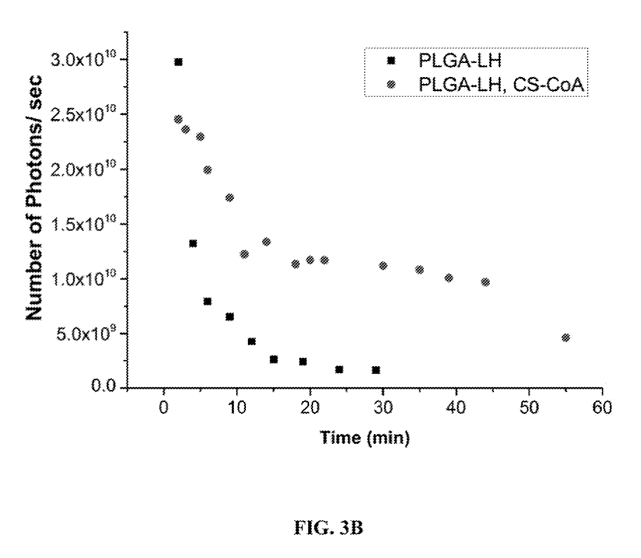

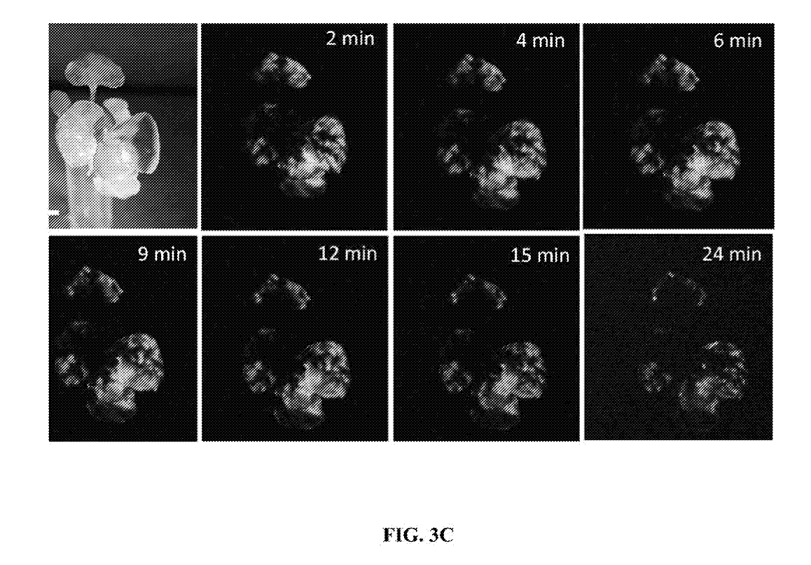

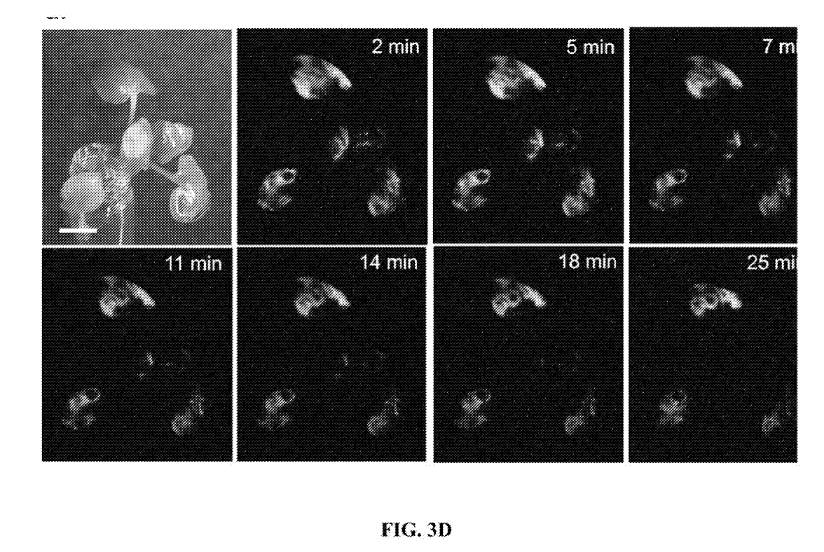

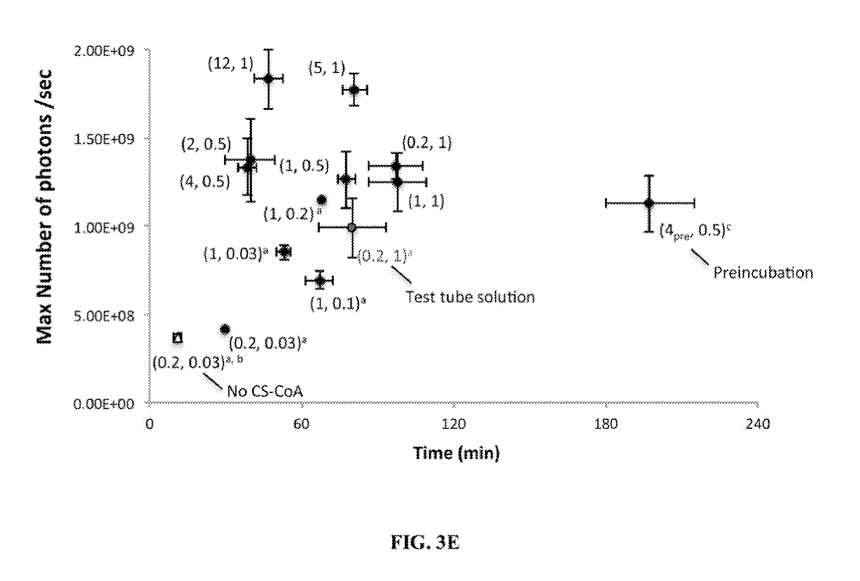

Additionally, the pressurization velocity appears to strongly affect the efficiency of PBIN. When 0.4 bar/s was applied to a spinach leaf (FIG. 2D(1)), infiltration was completed within seconds, however damage to the mesophyll was apparent, including ruptured cell membranes observed in fluorescence confocal microscopy (see FIG. 8). Leaf discs of spinach plants 3 week-olds were infiltrated with 30 mM HEPES buffer (pH 7.4) via PBIN at speed of 0.4 bar/s and cell membranes were stained with FM 464 fluorescent dye (red). Cell membranes are damaged. PBIN was successful at 0.04 bar/s applied (FIG. 2D (2), (3) and (6)) without such damage but notably was incomplete at pressurizations below 0.02 bar/s (FIG. 2D (4), (5)) despite reaching the same saturation pressure in all cases. Upon infiltration, the system of nanoparticles generates bright emission as high as 2.98×10¹⁰ photons/s. The decay in the luminescent intensity was strongly dependent on the incubation time of SNP-Luc within the living plants (FIG. 3A). SNP-Luc was infiltrated by LIN and kept in plant incubator for different time (30 min to 2 h) before free luciferin (0.1 mM) was infiltrated by LIN. Surprisingly, the initial number of photons is 4 to 7 times higher upon 1-2 hours compared to 30 minutes incubation, despite the anticipated loss of luciferase activity with a t_{1/2} measured at 2 hours in live cells. See, Ignowski, J. M. & Schaffer, D. V. Kinetic analysis and modeling of firefly luciferase as a quantitative reporter gene in live mammalian cells. Biotechnol Bioeng 86, 827-834, doi:10.1002/bit.20059 (2004), which is incorporated by reference in its entirety. A portion of SNP-Luc localizes within the stomata guard cells and leaf mesophyll cells, but the majority is retained within the leaf mesophyll to diffuse into the plant cells. Immediately following luciferin addition after infiltration of SNP-Luc, light emission appear suppressed to yield a shortened lifetime driven by mainly extracellular ATP of μM compared with cytosol ATP of mM. See Song, C. J., Steinebrunner, I., Wang, X., Stout, S. C. & Roux, S. J. Extracellular ATP induces the accumulation of superoxide via NADPH oxidases in Arabidopsis. Plant Physiol 140, 1222-1232, doi:10.1104/pp.105.073072 (2006), and Blatt, M. R. Electrical Characteristics of Stomatal Guard-Cells—the Contribution of Atp-Dependent, Electrogenic Transport Revealed by Current-Voltage and Difference-Current-Voltage Analysis. J Membrane Biol 98, 257-274, doi:Doi 10.1007/Bf01871188 (1987), each of which is incorporated by reference in its entirety.

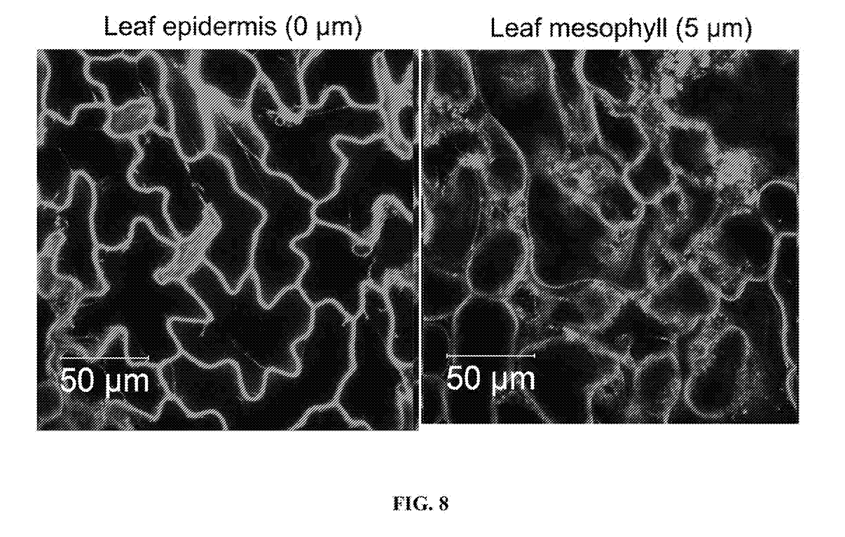

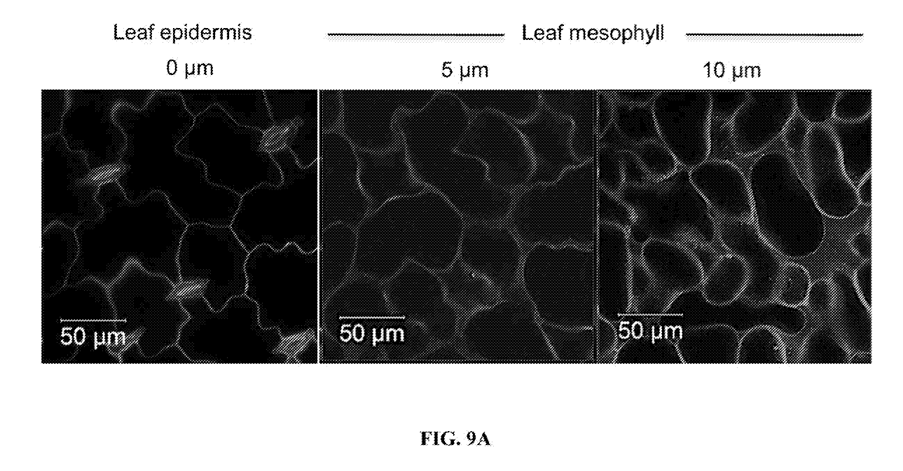

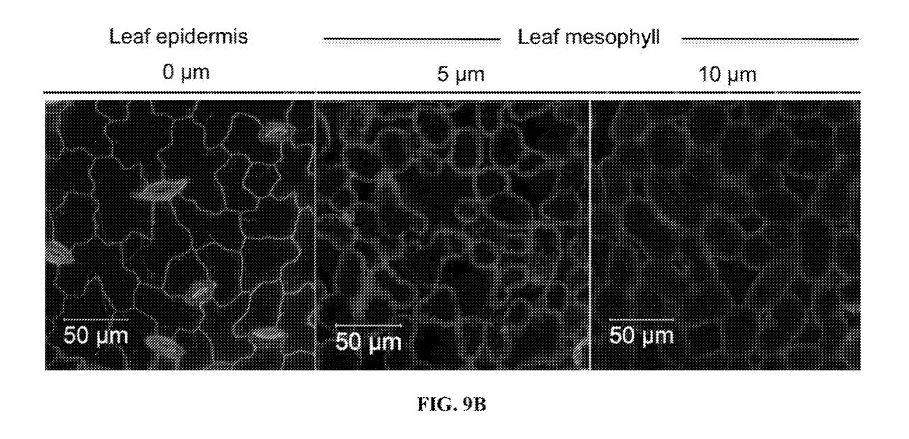

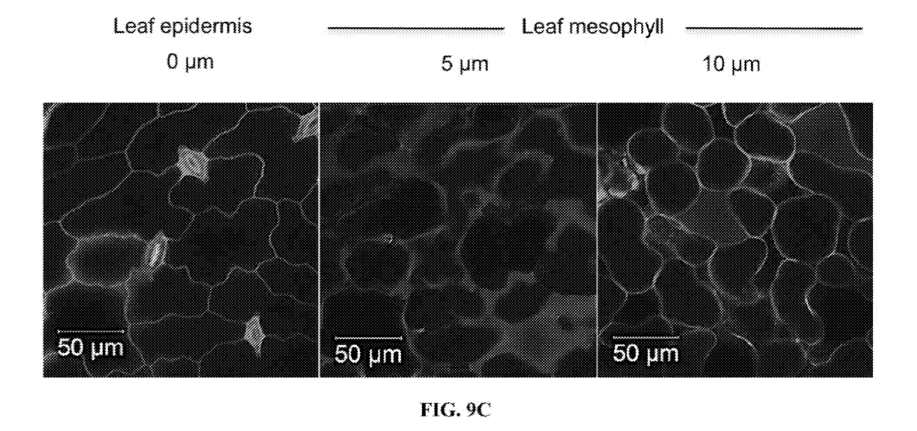

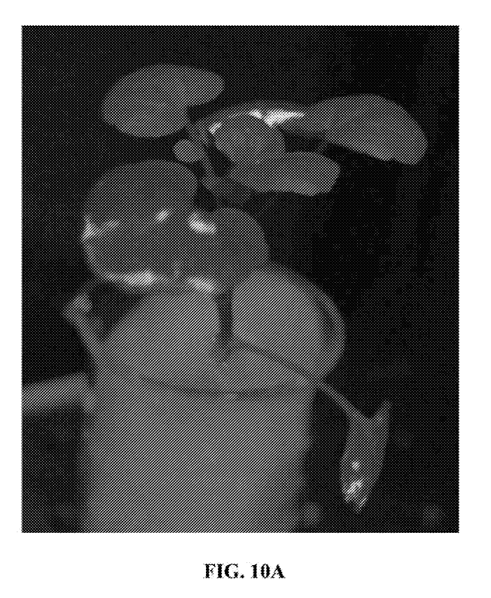

The influence of incubation time on the localization of SNPs is also apparent by fluorescent confocal micrographs (see FIGS. 9 and 10). In FIG. 9, leaf discs of spinach plants 3 weeks old were infiltrated with SNP-AF488 (green, 0.2 mg/mL) via LIN and stained with cell membrane FM 464 (red). Chloroplast emission is shown in blue. In FIG. 10A, whole watercress plant having even glowing patches after PBIN without pre-incubation of SNP-Luc. SNP luminescence images were overlaid with bright-field image by using Image J.

As PLGA-LH was infused using PBIN to the whole watercress plant, 6 cm tall, grown to maturity for 3 weeks, the initial burst of luciferin from PLGA-LH nanoparticles results in bright emission of 2.98×10¹⁰ photons/s (FIG. 3B) or 6% of a commercial LED. SNP-Luc was infiltrated by LIN 1 h in advance, and PLGA-LH alone or PLGA-LH and CS-CoA were infused into a watercress plant by PBIN. Despite the sharp drop in intensity after 5 minutes, light emission continues at moderate intensity over 30 minutes, sustained by a continuous supply of luciferin from the PLGA-LH (FIGS. 3B and 3C) in the mesophyll. To extend the illumination time, the third nanoparticle class, CS-CoA, was co-infiltrated with PLGA-LH by PBIN. CS-CoA is shown to dampen the initial intensity, but extend the duration substantially to more than 1 hour (FIGS. 3B and 3D). In FIG. 3E, a and b represent concentration of SNP-Luc (μM) and concentration of PLGA-LH2 (mM), respectively. Here, conserved conditions are as follows: 1) the concentration of CS-CoA was 625 μM except for d (no addition of CS-CoA for the white triangle, denoted (0.2, 0.03)^{d}), 2) The mixture of nanoparticles was applied by LIN, 3) ATP was supplied by root uptake except for a (the blue circle (0.2, 1)^{a}), and 4) 3-4 week old watercress plants were used. Maximum number of photons/sec was obtained from the first photo (analyzed by Image J). Photo was taken with a Nikon D5300 at a set of f/4.5, ISO 3200, and 30 sec exposure. The blue circle, denoted (0.2, 1)^{a}, is test tube solution consisting of 0.2 μM SNP-Luc, 1 mM PLGA-LH2, 625 μM CS-CoA and 0.5 mM ATP that showed 22 h-long light duration in vitro. Also, (4ₚᵣₑ, 0.5)^{c} means 4 μM SNP-Luc was infused into the all over the leaf, incubated for 1 hr, and then the mixture of 0.5 mM PLGA-LH₂ and CS-CoA was applied by LIN.

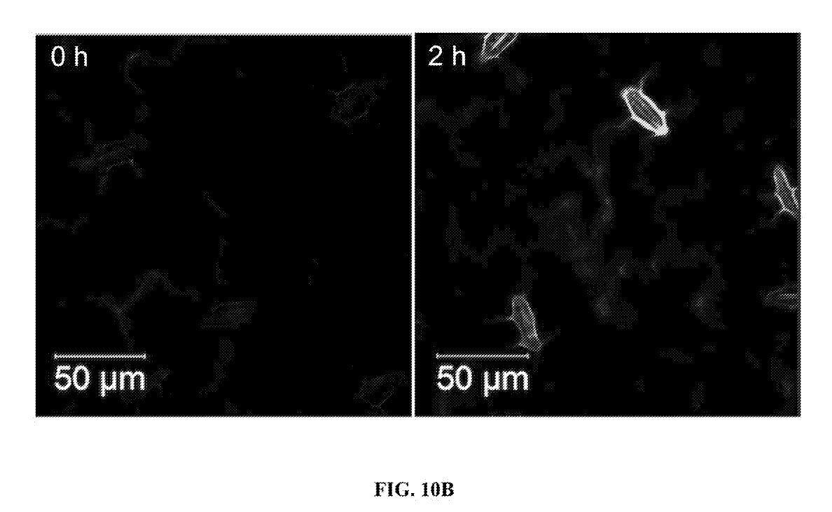

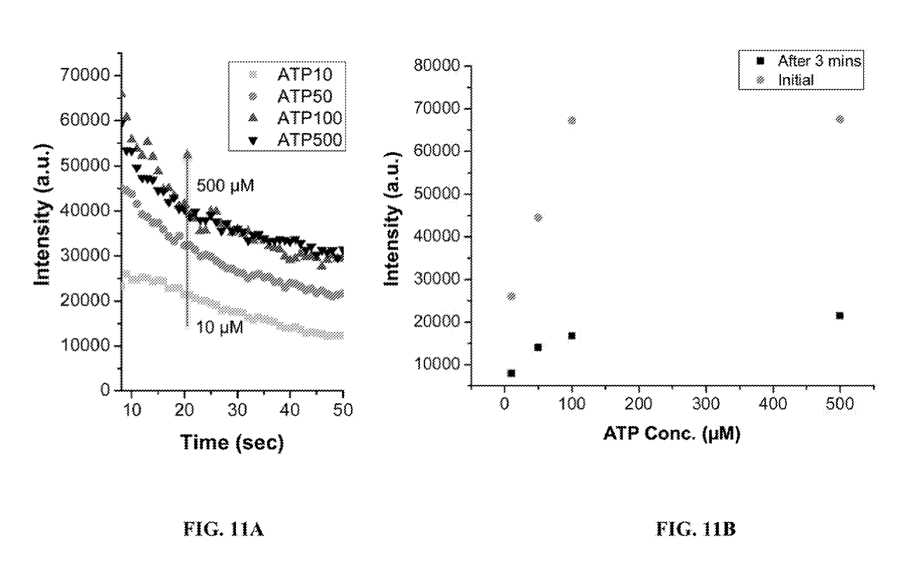

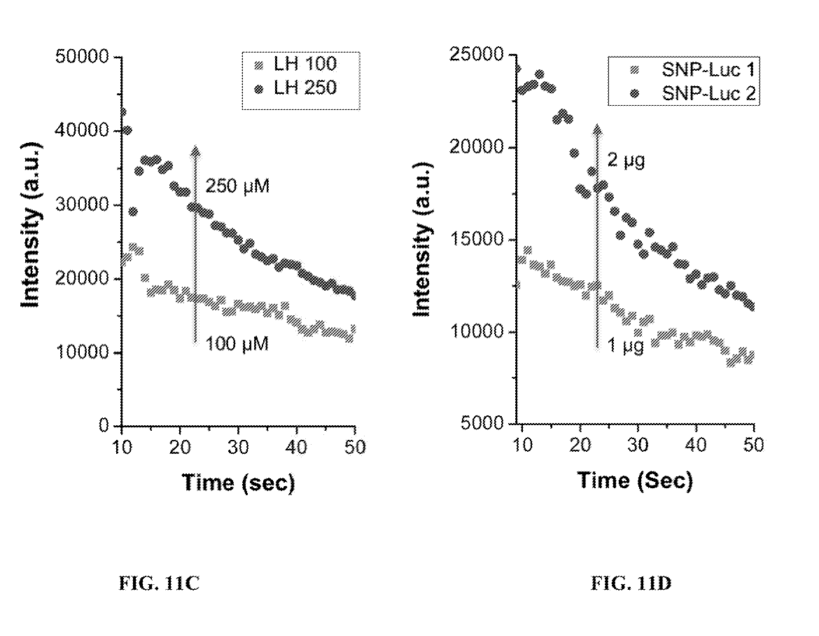

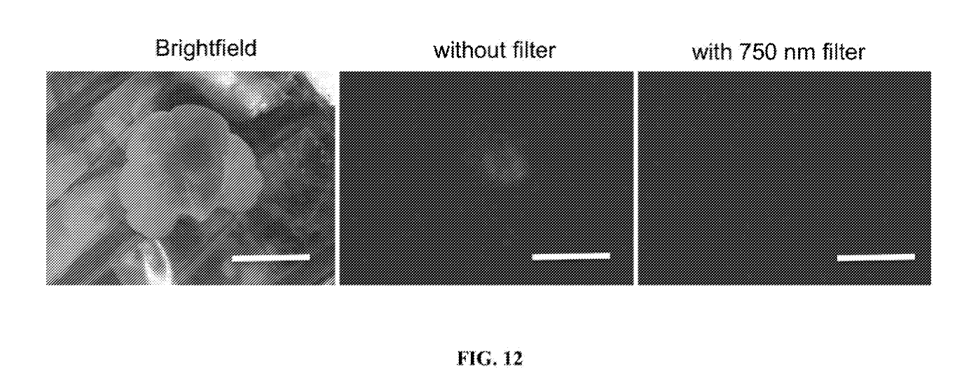

This initial brightness is predominantly dependent on the concentration of ATP and luciferase rather than luciferin in the living plants within the systems investigated in this work. FIG. 11 shows in vitro decay kinetics of nanoparticle complex luminescence. Fluorescence was measured by spectrofluorometer (Horiba Jobin Yvon, Fluorolog-3) with 0.1 s integration time, monitored at 560 nm. FIG. 11A shows luminescence intensity at different concentrations of ATP (10-500 μM) with 100 μM of luciferin and 2 μg of SNP-Luc, FIG. 11B shows luminescence intensity of the initial and 3 mins later at different concentration of ATP with 100 μM of luciferin and 2 μg of SNP-Luc. FIG. 11C shows luminescence intensity at different concentration of luciferin, 100 and 250 μM, with 10 μM of ATP and 2 μg of SNP-Luc, and FIG. 11D shows luminescence intensity at different SNP-Luc, 1 and 2 μg, with 10 μM of ATP and 100 μM of luciferin. As demonstrated by in vitro tests as well as living plants (see FIG. 11), luciferin requires a stoichiometric equivalence of ATP for oxidation by luciferase. Under conditions of low ATP concentration, this is often observed to be the bottleneck for light intensity. The system developed in this work will be a valuable tool for the study of ATP production in wild-type plants over extended durations, and in specific tissue compartments.

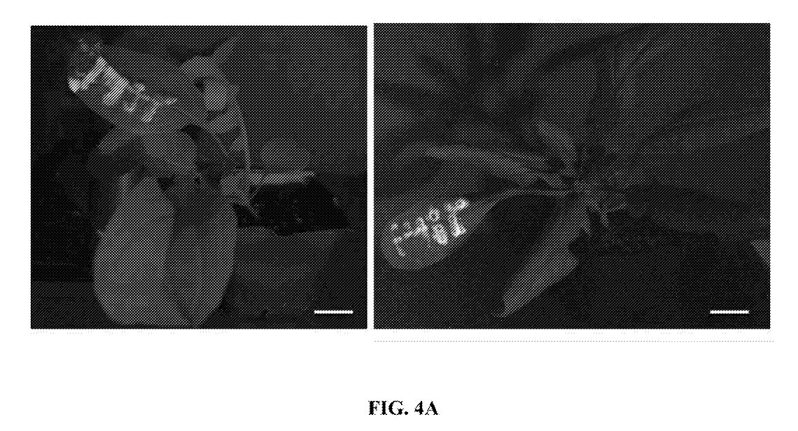

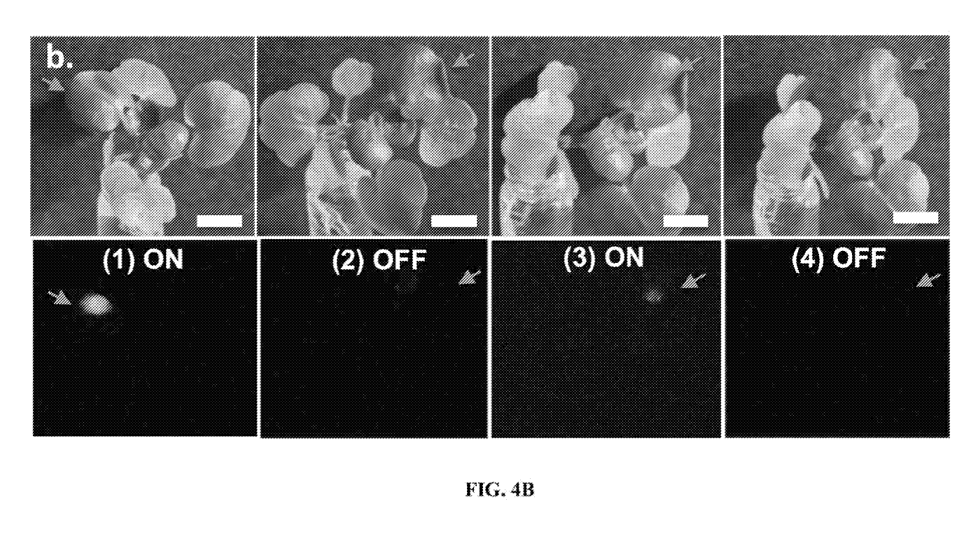

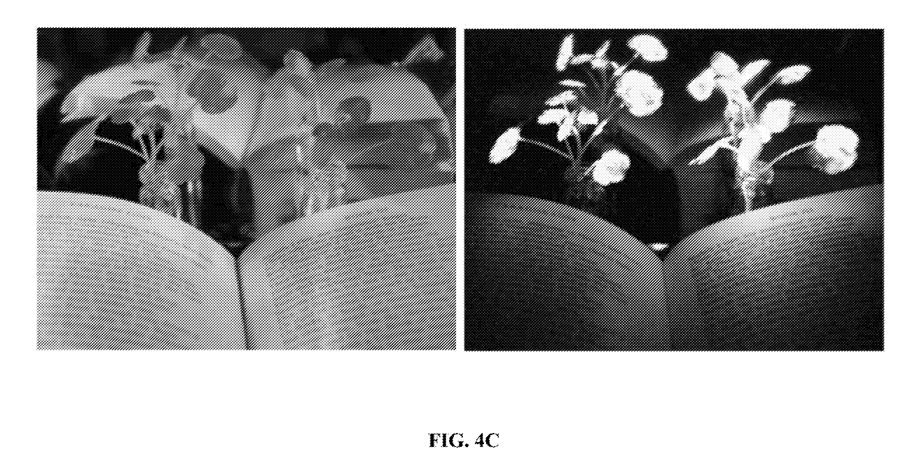

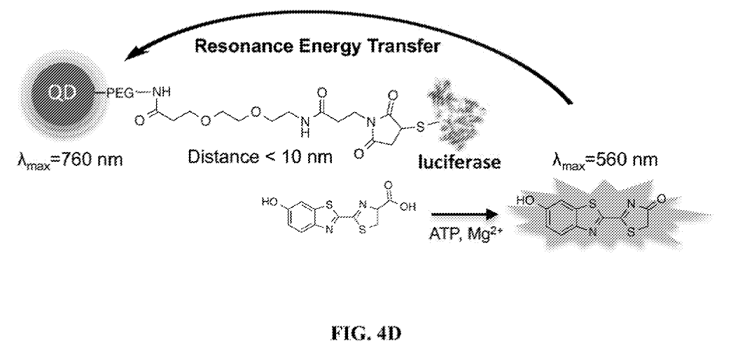

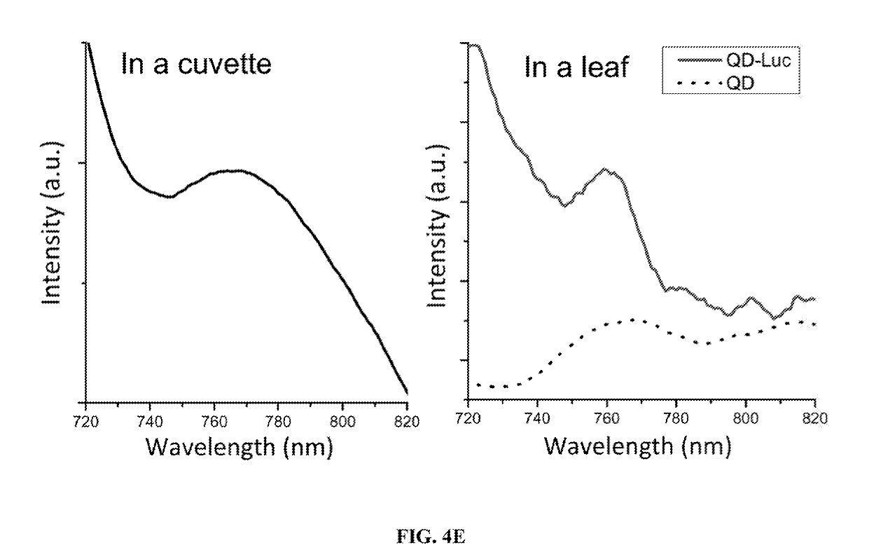

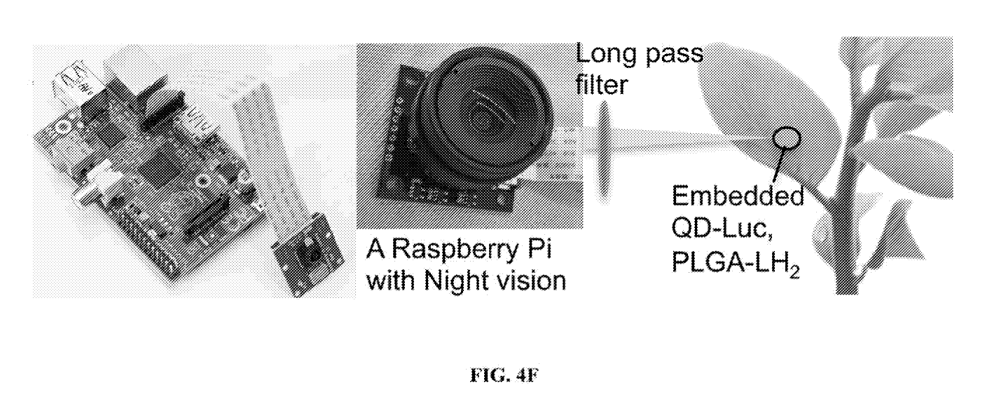

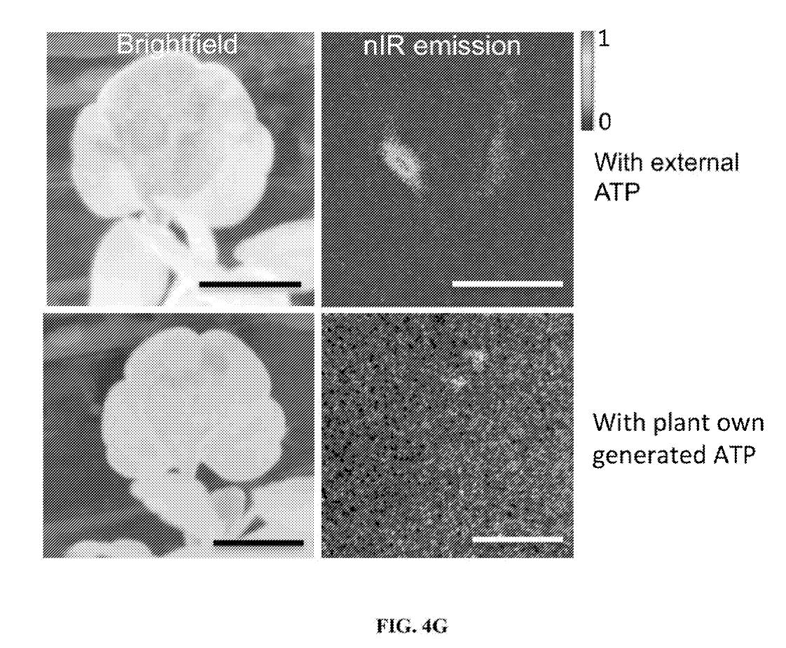

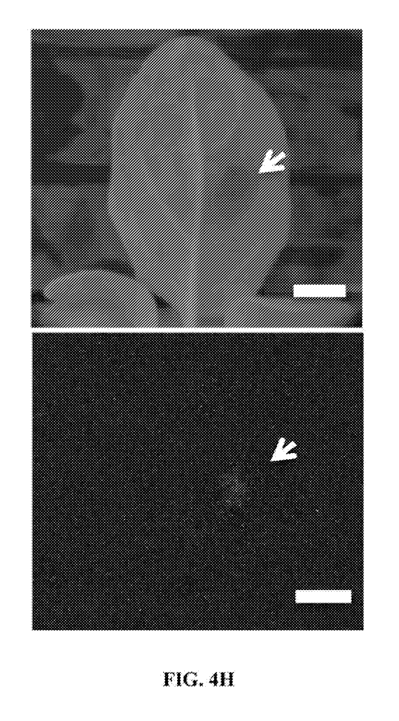

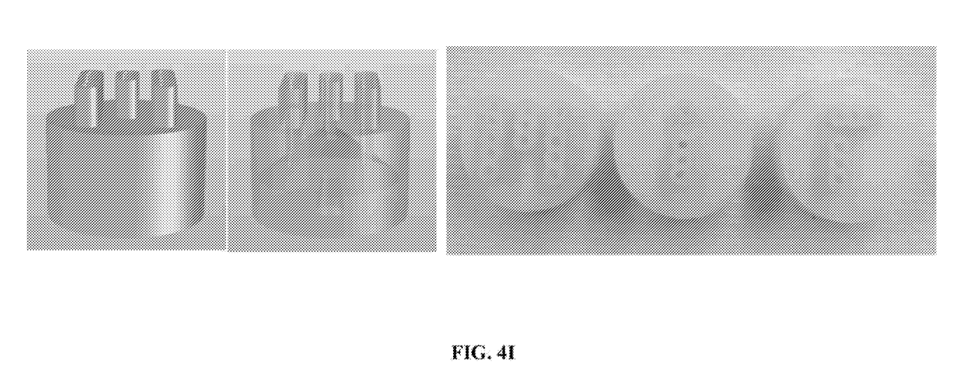

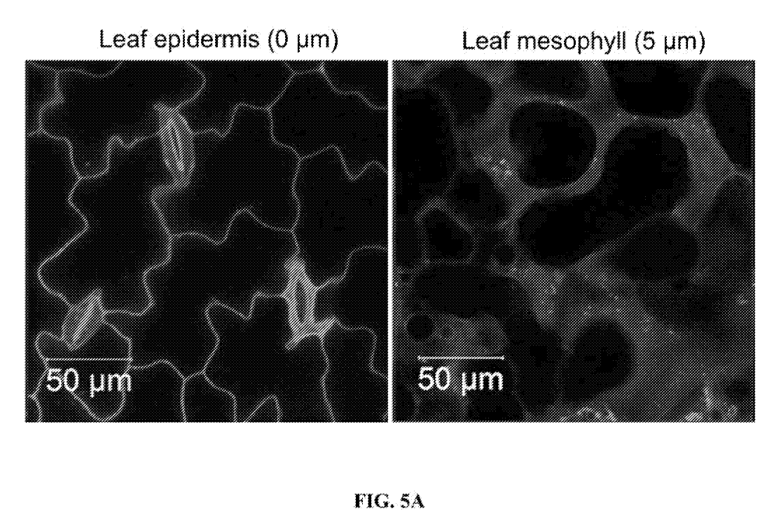

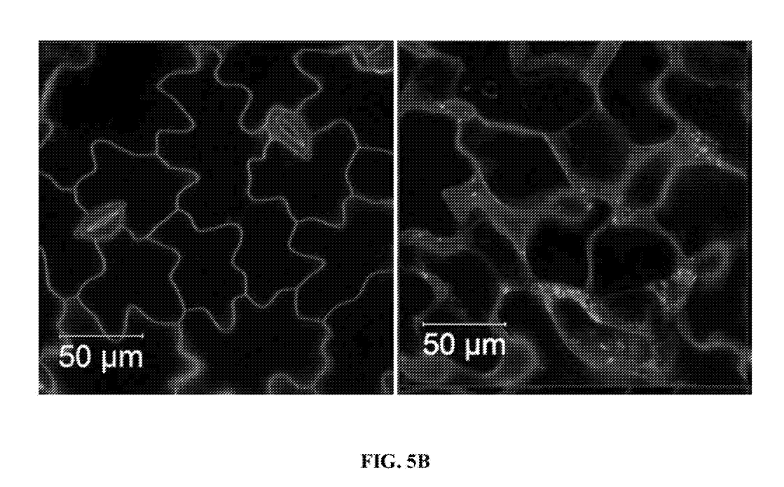

One advantage of this nanobionic approach is that the function of specific regions within tissues can be targeted, which is demonstrated by using an alternative to PBIN or leaf laminar infiltration of nanoparticles (LIN) through stomatal pores employed previously. See Giraldo, J. P. et al. Plant nanobionics approach to augment photosynthesis and biochemical sensing. Nat Mater 13, 400-408, doi:10.1038/nmat3890 (2014), which is incorporated by reference in its entirety. A syringe applicator in arbitrary letter shapes ‘M’, ‘I’, and ‘T’ with cone-shaped tapering was designed to minimize the loss of solution during pressurization (FIG. 4I). An illuminated ‘MIT’ logo was selectively infused into the leaves of two different species of plants, arugula and spinach (FIG. 4A). Fluorescence confocal micrographs of spinach leaves infiltrated by LIN shows that both leaf epidermal cell and eaf mesophyll cell regions showed similar nanoparticles distribution (FIG. 5B). Some nanoparticles are located in guard cells, but mostly in air spaces surrounding sponge mesophyll cells. Cell membranes are intact The ability to easily modify wild-type plants is a notable advantage of this nanobionic approach.

Another advantage of such an approach is that it is possible to shift the light emission to other wavelengths using resonant energy transfer to a semiconductor nanocrystal. A wavelength shift was demonstrated from the luciferin emission at 560 nm to the near infrared at 760 nm with 10 nm polyethylene glycol-capped CdSe quantum dots (FIG. 4D). The shifted emission at 760 nm was clearly shown in the cuvette containing mixture of luciferase conjugated quantum dots (QD-Luc), luciferin, and ATP and in a watercress leaf, which were measured by spectrofluorometer without laser excitation (FIG. 4E). When the QD-Luc was infused into living plants, a strong nIR emission signal without external laser excitation was easily detected using a simple Raspberry Pi CCD camera, equivalent to typical smart phone hardware at 6 sec exposure (FIG. 4G). The emission can be further enhanced after the addition of ATP, however nIR emission is clearly detectable using the plant's own ATP exclusively. FIG. 4H shows that QD-Luc was embedded in an arugula plant, and luciferin was added through root uptake. This demonstration illustrates the potential for ambient IR communications from a plant system, with future work to address control of modulation and multiplexing for more complex communications to external electronic devices.

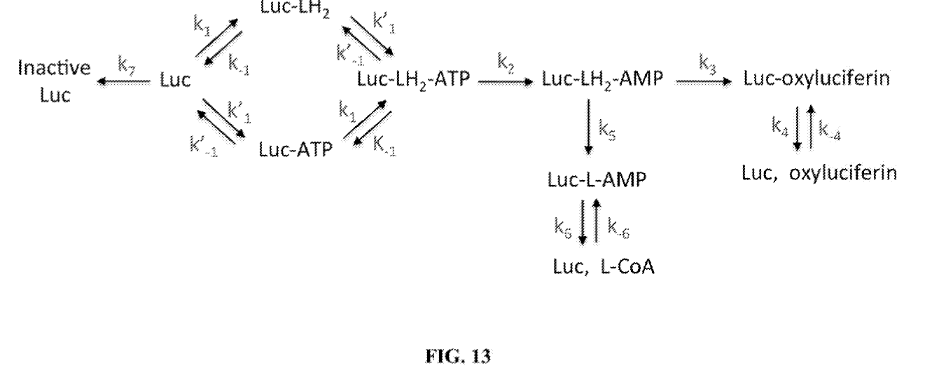

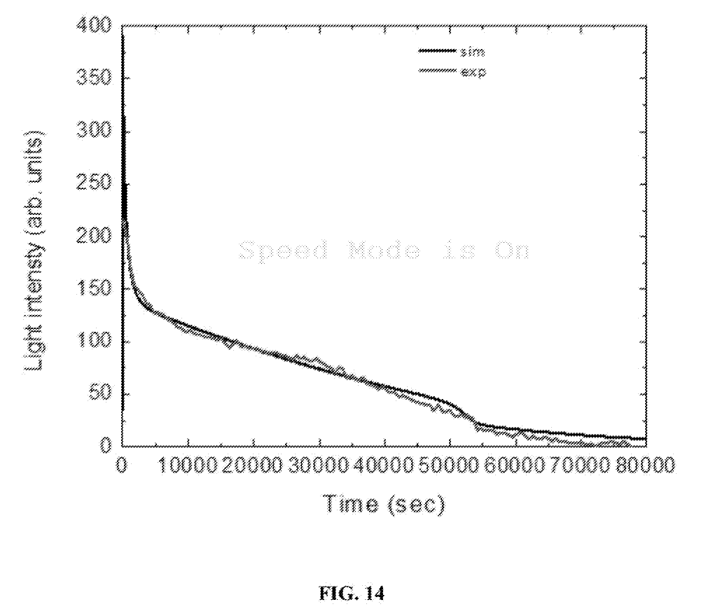

A kinetic model for luciferase-luciferin reaction was constructed including a role of coenzyme A (CoA) (FIG. 13). The constants of each reaction steps were obtained from previous reports. See, DeLuca, M. and W. D. McElroy, Kinetics of the firefly luciferase catalyzed reactions. Biochemistry, 1974. 13(5): p. 921-925, Lembert, N. and L. A. Idahl, Regulatory effects of ATP and luciferin on firefly luciferase activity. Biochemical Journal, 1995. 305(Pt 3): p. 929-93, Agah, A., et al., A multi-enzyme model for pyrosequencing. Nucleic Acids Research, 2004. 32(21): p. e166, Fraga, H., et al., Coenzyme A affects firefly luciferase luminescence because it acts as a substrate and not as an allosteric effector. FEBS Journal, 2005. 272(20): p. 5206-5216, and da Silva, L. P. and J. C. G. Esteves da Silva, Kinetics of inhibition of firefly luciferase by dehydroluciferyl-coenzyme A, dehydroluciferin and 1-luciferin. Photochemical & Photobiological Sciences, 2011. 10(6): p. 1039-1045, each of which is incorporated by reference in its entirety. Based on the model constructed by Agah et al., the model was expanded to include the dark reaction, which produced a strong luciferase inhibitor (dehydroluciferin) resulting in inactivation of luciferase, and reactivation of luciferase by thiolytic reaction using CoA. See, Agah, A., et al., A multi-enzyme model for pyrosequencing. Nucleic Acids Research, 2004. 32(21): p. e166, which is incorporated by reference in its entirety. All reactions are either first or second order, while the thiolytic reaction of CoA follows Michaelis-Menten kinetics. A method of non-linear regression is used to obtain the great fit to our measurements. The initial constants from literature, optimized values of free luciferase and immobilized luciferase (SNP-Luc) in this study were summarized in Table 1.

TABLE 1
Comparison between starting and optimized constants for
a kinetic model

| | Starting model: | | | |
|---|---|---|---|---|
| Constant | data from Agah et al. | | Optimized values | |
| k₁ | 4.32 10⁻⁵ | nM⁻¹ s⁻¹ | 3 10⁻⁵ | nM⁻¹ s⁻¹ |
| k₋₁ | 10⁻³ | nM⁻¹ s⁻¹ | 5 10⁻⁴ | nM⁻¹ s⁻¹ |
| k′₁ | 3.5 | s⁻¹ | 0.1 | s⁻¹ |
| k′₋₁ | 10 | s⁻¹ | 0.1 | s⁻¹ |
| k₂ | 19.2 | s⁻¹ | 30 | s⁻¹ |
| k₃ | 0.96 | s⁻¹ | 0.04 | s⁻¹ |
| k₄ | 0.1 | s⁻¹ | 1.6 | s⁻¹ |
| k₋₄ | — | | 0.032 | nM⁻¹ s⁻¹ |
| k₅ | — | | 0.005 | s⁻¹ |
| k₆ | — | | 45 10⁻⁵ | nM⁻¹ s⁻¹ |
| k₋₆ | — | | 1 | nM⁻¹ s⁻¹ |
| k₇ | 2.6 10⁻⁵ | s⁻¹ | 5 10⁻⁵ | s⁻¹ |

The disclosed nanobionic light emitting plants with record levels of both brightness and luminescent lifetime, tissue specific patterning and wavelength modulation through resonant energy transfer open possibilities towards useful tools to create plants with non-native functions, photonic sources for indirect lighting and nIR communications, as well as to contribute to the fundamental study of plant biology in a variety of wild-type plants.

As used herein, the term “nanoparticle” refers to articles having at least one cross-sectional dimension of less than about 1 micron. A nanoparticle can also be referred to as a “nanostructure.” A nanoparticle can have at least one cross-sectional dimension of less than about 500 nm, less than about 250 nm, less than about 100 nm, less than about 75 nm, less than about 50 nm, less than about 25 nm, less than about 10 nm, less than 5 nm, or, in some cases, less than about 1 nm. Examples of nanoparticle include nanotubes (e.g., carbon nanotubes), nanowires (e.g., carbon nanowires), graphene, and quantum dots, among others. In some embodiments, the nanoparticle can include a fused network of atomic rings, the atomic rings comprising a plurality of double bonds.

A nanoparticle can be a photoluminescent nanoparticle. A “photoluminescent nanoparticle,” as used herein, refers to a class of nanoparticles that are capable of exhibiting photoluminescence. In some cases, photoluminescent nanoparticles can exhibit fluorescence. In some instances, photoluminescent nanoparticles exhibit phosphorescence. Examples of photoluminescent nanoparticles suitable for use include, but are not limited to, single-walled carbon nanotubes (SWCNTs), double-walled carbon nanotubes (DWCNTs), multi-walled carbon nanotubes (MWCNTs), semi-conductor quantum dots, semi-conductor nanowires, and graphene, among others.

A variety of nanoparticles can be used. Sometimes a nanoparticle can be a carbon-based nanoparticle. As used herein, a “carbon-based nanoparticle” can include a fused network of aromatic rings wherein the nanoparticle includes primarily carbon atoms. In some instances, a nanoparticle can have a cylindrical, pseudo-cylindrical, or horn shape. A carbon-based nanoparticle can include a fused network of at least about 10, at least about 50, at least about 100, at least about 1000, at least about 10,000, or, in some cases, at least about 100,000 aromatic rings. A carbon-based nanoparticle may be substantially planar or substantially non-planar, or may include a planar or non-planar portion. A carbon-based nanoparticle may optionally include a border at which the fused network terminates. For example, a sheet of graphene includes a planar carbon-containing molecule including a border at which the fused network terminates, while a carbon nanotube includes a non-planar carbon-based nanoparticle with borders at either end. In some cases, the border may be substituted with hydrogen atoms. In some cases, the border may be substituted with groups comprising oxygen atoms (e.g., hydroxyl).

In some embodiments, a nanoparticle can include or be a nanotube. The term “nanotube” is given its ordinary meaning in the art and can refer to a substantially cylindrical molecule or nanoparticle including a fused network of primarily six-membered rings (e.g., six-membered aromatic rings). In some cases, a nanotube can resemble a sheet of graphite formed into a seamless cylindrical structure. It should be understood that a nanotube may also include rings or lattice structures other than six-membered rings. Typically, at least one end of the nanotube may be capped, i.e., with a curved or non-planar aromatic group. A nanotube may have a diameter of the order of nanometers and a length on the order of microns, tens of microns, hundreds of microns, or millimeters, resulting in an aspect ratio greater than about 100, about 1000, about 10,000, or greater. In some embodiments, a nanotube can have a diameter of less than about 1 micron, less than about 500 nm, less than about 250 nm, less than about 100 nm, less than about 75 nm, less than about 50 nm, less than about 25 nm, less than about 10 nm, or, in some cases, less than about 1 nm.

In some embodiments, a nanotube may include a carbon nanotube. The term “carbon nanotube” can refer to a nanotube including primarily carbon atoms. Examples of carbon nanotubes can include single-walled carbon nanotubes (SWNTs), double-walled carbon nanotubes (DWNTs), multi-walled carbon nanotubes (MWNTs) (e.g., concentric carbon nanotubes), inorganic derivatives thereof, and the like. In some embodiments, a carbon nanotube can be a single-walled carbon nanotube. In some cases, a carbon nanotube can be a multi-walled carbon nanotube (e.g., a double-walled carbon nanotube).

In some embodiments, a nanoparticle can include non-carbon nanoparticles, specifically, non-carbon nanotubes. Non-carbon nanotubes may be of any of the shapes and dimensions outlined above with respect to carbon nanotubes. A non-carbon nanotube material may be selected from polymer, ceramic, metal and other suitable materials. For example, a non-carbon nanotube may include a metal such as Co, Fe, Ni, Mo, Cu, Au, Ag, Pt, Pd, Al, Zn, or alloys of these metals, among others. In some instances, a non-carbon nanotube may be formed of a semi-conductor such as, for example, Si. In some cases, a non-carbon nanotube may include a Group II-VI nanotube, wherein Group II includes Zn, Cd, and Hg, and Group VI includes O, S, Se, Te, and Po. In some embodiments, a non-carbon nanotube may include a Group III-V nanotube, wherein Group III includes B, Al, Ga, In, and Tl, and Group V includes N, P, As, Sb, and Bi. As a specific example, a non-carbon nanotube may include a boron-nitride nanotube. In other embodiments, the nanoparticle can be a ceramic, for example, a metal oxide, metal nitride, metal boride, metal phosphide, or metal carbide. In this example, the metal can be any metal, including Group I metal, Group II metal, Group III metal, Group IV metal, transition metal, lanthanide metal or actinide metal. For example, the ceramic can include one or more of metal, for example, Li, Na, K, Rb, Cs, Be, Mg, Ca, Sr, Ba, Sc, Y, La, Ce, Pr, Nd, Pm, Sm, Eu, Gd, Tb, Dy, Ho, Er, Tm, Yb, Lu, Ti, Zr, Hf, V, Nb, Ta, Cr, Mo, W, Mn, Tc, Re, Fe, Ru, Os, Co, Rh, Ir, Ni, Pd, Pt, Cu, Ag, Su, Zn, Cd, Hg, Al, Ga, In, Tl, Si, Ge, Sn, Pb or Bi.

In some embodiments, a nanotube may include both carbon and another material. For example, in some cases, a multi-walled nanotube may include at least one carbon-based wall (e.g., a conventional graphene sheet joined along a vector) and at least one non-carbon wall (e.g., a wall comprising a metal, silicon, boron nitride, etc.). In some embodiments, the carbon-based wall may surround at least one non-carbon wall. In some instances, a non-carbon wall may surround at least one carbon-based wall.

The term “quantum dot” is given its normal meaning in the art and can refer to semi-conducting nanoparticles that exhibit quantum confinement effects. Generally, energy (e.g., light) incident upon a quantum dot can excite the quantum dot to an excited state, after which, the quantum dot can emit energy corresponding to the energy band gap between its excited state and its ground state. Examples of materials from which quantum dots can be made include PbS, Pb Se, CdS, CdSe, ZnS, and ZnSe, among others.

A photoluminescent nanoparticle can be, in some cases, substantially free of dopants, impurities, or other non-nanoparticle atoms. For example, in some embodiments, a nanoparticle can include a carbon nanoparticle that is substantially free of dopants. As a specific example, in some embodiments, a nanoparticle can include single-walled carbon nanotube that contains only aromatic rings (each of which contains only carbon atoms) within the shell portion of the nanotube. In other words, a nanoparticle can consist essentially of a single material, for example, carbon.

In some embodiments, a photoluminescent nanoparticle may emit radiation within a desired range of wavelengths. For example, in some cases, a photoluminescent nanoparticle may emit radiation with a wavelength between about 750 nm and about 1600 nm, or between about 900 nm and about 1400 nm (e.g., in the near-infrared range of wavelengths). In some embodiments, a photoluminescent nanoparticle may emit radiation with a wavelength within the visible range of the spectrum (e.g., between about 400 nm and about 700 nm).

In some embodiments, a photoluminescent nanoparticle may be substantially free of covalent bonds with other entities (e.g., other nanoparticles, a current collector, the surface of a container, a polymer, an analyte, etc.). The absence of covalent bonding between a photoluminescent nanoparticle and another entity may, for example, preserve the photoluminescent character of the nanoparticle. In some cases, single-walled carbon nanotubes or other photoluminescent nanoparticles may exhibit modified or substantially no fluorescence upon forming a covalent bond with another entity (e.g., another nanoparticle, a current collector, a surface of a container, and the like).

In some embodiments, a nanoparticle can include cerium oxide. A nanoparticle including cerium oxide can be referred to as nanoceria. A nanoparticle can be cerium oxide. A nanoparticle can also be conjugated to at least one cerium oxide nanoparticle. Conjugation can be direct or indirect. Conjugation can also be through a covalent bond, ionic bond or van der Waals interaction. A nanoparticle can be cross-linked with at least one cerium oxide nanoparticle, more specifically, cross-linked using via carbodiimide chemistry. In one example, a carbodiimide agent N-ethyl-N′-(3-dimethylaminopropyl) carbodiimide hydrochloride (EDC) can be used.

A nanoparticle can be strongly cationic or anionic. Strongly cationic or anionic can mean that the nanoparticle (or other element) has a high magnitude of the zeta potential. For example, the nanoparticle can have a zeta potential of less than −10 mV or greater than 10 mV. In preferred embodiments, the nanoparticle can have a zeta potential of less than −20 mV or greater than 20 mV, a zeta potential of less than −30 mV or greater than 30 mV, or a zeta potential of less than −40 mV or greater than 40 mV.

A nanoparticle can include a coating or be suspended in a coating with a high magnitude of the zeta potential. A coating can be a polymer. A variety of polymers may be used in association with the embodiments described herein. In some cases, the polymer may be a polypeptide. In some embodiments, the length and/or weight of the polypeptide may fall within a specific range. For example, the polypeptide may include, in some embodiments, between about 5 and about 50, or between about 5 and about 30 amino acid residues. In some cases, the polypeptide may have a molecular weight of between about 400 g/mol and about 10,000 g/mol, or between about 400 g/mol and about 600 g/mol. Examples of protein polymers can include glucose oxidase, bovine serum albumin and alcohol dehydrogenase.

A polymer may include a synthetic polymer (e.g., polyvinyl alcohol, poly(acrylic acid), poly(ethylene oxide), poly(vinyl pyrrolidinone), poly(allyl amine), poly(2-vinylpyridine), poly(maleic acid), and the like), in some embodiments.

In some embodiments, the polymer may include an oligonucleotide. The oligonucleotide can be, in some cases, a single-stranded DNA oligonucleotide. The single-stranded DNA oligonucleotide can, in some cases, include a majority (>50%) A or T nucleobases. In some embodiments, single-stranded DNA oligonucleotide can include more than 75%, more than 80%, more than 90%, or more than 95% A or T nucleobases. In some embodiments, the single-stranded DNA oligonucleotide can include a repeat of A and T. For example, a oligonucleotide can be, in some cases, at least 5, at least 10, at least 15, between 5 and 25, between 5 and 15, or between 5 and 10 repeating units, in succession, of (GT) or (AT). Repeating units can include at least 2 nucleobases, at least 3 nucleobases, at least 4 nucleobases, at least 5 nucleotides long. The nucleobases described herein are given their standard one-letter abbreviations: cytosine (C), guanine (G), adenine (A), and thymine (T).

In some embodiments, the polymer can include a polysaccharide such as, for example, dextran, pectin, hyaluronic acid, hydroxyethylcellulose, amylose, chitin, or cellulose.

In preferred embodiments, the interaction between a polymer and a nanoparticle can be non-covalent (e.g., via van der Waals interactions); however, a polymer can covalently bond with a nanoparticle. In some embodiments, the polymer may be capable of participating in a pi-pi interaction with the nanostructure. A pi-pi interaction (a.k.a., “pi-pi stacking”) is a phenomenon known to those of ordinary skill in the art, and generally refers to a stacked arrangement of molecules adopted due to interatomic interactions. Pi-pi interactions can occur, for example, between two aromatic molecules. If the polymer includes relatively large groups, pi-pi interaction can be reduced or eliminated due to steric hindrance. Hence, in certain embodiments, the polymer may be selected or altered such that steric hindrance does not inhibit or prevent pi-pi interactions. One of ordinary skill in the art can determine whether a polymer is capable or participating in pi-pi interactions with a nanostructure.

The polymer may be strongly cationic or anionic, meaning that the polymer has a high magnitude of the zeta potential. For example, the polymer can have a zeta potential of less than −10 mV or greater than 10 mV, less than −20 mV or greater than 20 mV, less than −30 mV or greater than 30 mV, or less than −40 mV or greater than 40 mV.

A nanoparticle can be contained within a mesophyll or stomata guard cells, as demonstrated more fully herein. A nanoparticle can traverse and/or localize within the outer membrane layer (i.e., lipid bilayer). The process can be complete and/or irreversible. Because other organelles include an outer membrane layer (i.e., lipid bilayer), a nanoparticle can be contained within other organelles. For example, other organelles that a nanoparticle can be introduced into can include a nucleus, endoplasmic reticulum, Golgi apparatus, chloroplast, chromoplast, gerontoplast, leucoplast, lysosome, peroxisome, glyoxysome, endosome, mitochondria or vacuole.

Thylakoids are a membrane-bound compartment inside a chloroplast. Cyanobacteria can also include thylakoids. In some embodiments, a nanoparticle can be associated with a thylakoid membrane within a chloroplast, cyanobacteria or other photocatalytic cell or organelle.

A nanoparticle can be contained within a photocatalytic unit, most preferably, including an outer lipid membrane (i.e., lipid bilayer). A photocatalytic unit can be a structure capable of performing photosynthesis or photocatalysis, preferably a cell or an organelle capable of performing photosynthesis or photocatalysis. For example, a photocatalytic unit can be a chloroplast, a cyanobacteria, or a bacterial species selected from the group consisting of Chlorobiacea spp., a Chromaticacea spp. and a Rhodospirillacae spp.

An organelle can be part of a cell, a cell can be part of a tissue, and a tissue can be part of an organism. For example, a nanoparticle can be contained within a cell of a leaf of a plant. More to the point, a cell can be intact. In other words, the organelle may not be an isolated organelle, but rather, the organelle can be contained within the outer lipid membrane of a cell.

A nanoparticle that is independent of an organelle or cell can be free of lipids. An outer lipid membrane can enclose or encompass an organelle or cell. As the nanoparticle traverses the outer lipid membrane of an organelle or cell, lipids from the outer lipid membrane can associate or coat the nanoparticle. As a result, a nanoparticle inside the outer lipid membrane of an organelle or cell can be associated with or coated with lipids that originated in the organelle or cell.

Transport of a nanoparticle into an organelle or a cell can be an active process. In some cases, transport across the outer lipid membrane can be dependent on the pressure, temperature or light conditions.

Transport of a nanoparticle into an organelle or a cell can be a passive process. In some cases, transport across the outer lipid membrane can be independent of the pressure, temperature or light conditions.

Embedding a nanoparticle within an organelle or cell can be useful for monitoring the activity of the organelle or cell. For example, a nanoparticle, preferably a photoluminescent nanoparticle, can be introduced into the organelle or cell. Measurements of the photoluminescence of a photoluminescent nanoparticle can provide information regarding a stimulus within an organelle or cell. Measurements of the photoluminescence of a photoluminescent nanoparticle can be taken at a plurality of time points. A change in the photoluminescence emission between a first time point and a second time point can indicate a change in a stimulus within the organelle or cell.

In some embodiments, a change in the photoluminescence emission can include a change in the photoluminescence intensity, a change in an emission peak width, a change in an emission peak wavelength, a Raman shift, or combination thereof. One of ordinary skill in the art would be capable of calculating the overall intensity by, for example, taking the sum of the intensities of the emissions over a range of wavelengths emitted by a nanoparticle. In some cases, a nanoparticle may have a first overall intensity, and a second, lower overall intensity when a stimulus changes within the organelle or cell. In some cases, a nanoparticle may emit a first emission of a first overall intensity, and a second emission of a second overall intensity that is different from the first overall intensity (e.g., larger, smaller) when a stimulus changes within the organelle or cell.

A nanoparticle may, in some cases, emit an emission of radiation with one or more distinguishable peaks. One of ordinary skill in the art would understand a peak to refer to a local maximum in the intensity of the electromagnetic radiation, for example, when viewed as a plot of intensity as a function of wavelength. In some embodiments, a nanoparticle may emit electromagnetic radiation with a specific set of peaks. In some cases, a change in a stimulus may cause the nanoparticle to emit electromagnetic radiation including one or more peaks such that the peaks (e.g., the frequencies of the peaks, the intensity of the peaks) may be distinguishable from one or more peaks prior to the change in stimulus. In some cases, the change in a stimulus may cause the nanoparticle to emit electromagnetic radiation comprising one or more peaks such that peaks (e.g., the frequencies of the peaks, the intensity of the peaks) are distinguishable from the one or more peaks observed prior to the change in the stimulus. When the stimulus is the concentration of an analyte, the frequencies and/or intensities of the peaks may, in some instances, allow one to determine the analyte interacting with the nanoparticle by, for example, producing a signature that is unique to a particular analyte that is interacting with the nanoparticle. Determination of a specific analyte can be accomplished, for example, by comparing the properties of the peaks emitted in the presence of the analyte to a set of data (e.g., a library of peak data for a predetermined list of analytes).

A stimulus can include the pH of the organelle or cell. A change in the pH can be an increase or decrease in the pH.

A stimulus can include a modification of an analyte. For example, an analyte may be oxidized or reduced. In other examples, an analyte can be ionized. In another example, an analyte can include an ether, ester, acyl, or disulfide or other derivative.

A stimulus can include the concentration of an analyte. An analyte can include a reactive oxygen species, for example, hydrogen peroxide, superoxide, nitric oxide, and a peroxidase. Alternatively, an analyte can be carbon dioxide, adenosine triphosphate (ATP), nicotinamide adenine dinucleotide phosphate (NADP⁺ or NADPH), or oxygen. In some instances, the concentration of the analyte may be relatively low (e.g., less than about 100 micromolar, less than about 10 micromolar, less than about 1 micromolar, less than about 100 nanomolar, less than about 10 nanomolar, less than about 1 nanomolar, or about a single molecule of the analyte). In some cases, the concentration of an analyte may be zero, indicating that no analyte is present.

Functionalized nanotubes can be useful in many areas. In one embodiment, nanotubes can be functionalized in different ways to serve as sensors for harmful compounds. To detect explosives, bombolitin-functionalized nanotubes can be infused into the leaves of the plant. Bombolitin is a unique peptide which allows for recognition of nitroaromatics, the key compounds in many explosives. Therefore, a plant with bombolitin-functionalized nanoutbes can recognize the nitroaromatics from explosives. Using stand-off devices for detecting the spectral shift, semiconducting SWNT and SWNT-based sensors within plants can be imaged from a distance of several meters to hundreds of metters, for example, from 3-10 meters, 10-40 meters, 40-100 meters, 100-500 meters, or 500-1000 meters, and even from a satellite.

A light emitting compound immobilized on nanoparticles can be introduced to a green plant to make an autoluminescent plant. In one embodiment, co-immobilization of luciferase and luciferin on mesoporous silica nanoparticles can make autoluminescent plants without genetic modification. Immobilizing luciferase on silica nanoparticles with ATP in plant leaves can make the luminescence reactions to glow for longer time durations compared to free luciferase in a leaf.

The interface between plant organelles and non-biological nanoparticles has the potential to impart the former with new and enhanced functions. For example, this nanobionic approach can yield chloroplasts that possess enhanced photosynthetic activity both ex vivo and in vivo, are more stable to reactive oxygen species ex vivo, and allow real time information exchange via embedded nanosensors for free radicals in plants. Accordingly, there is a need for nanoparticles that can interface with organelles, specifically, plant organelles ex vivo and in vivo to enable novel or enhanced functions. Similarly, there is a need for nanoparticles that can interface with intact photosynthetic organisms or intact cells of photosynthetic organisms ex vivo and in vivo to enable novel or enhanced functions. For example, the assembly of nanoparticle complexes within chloroplast photosynthetic machinery has the potential to enhance solar energy conversion through augmented light reactions of photosynthesis and ROS scavenging while imparting novel sensing capabilities to living plants. In optical communications, the light generated by the plant can be used to power or excite optical sensors also in the plant.

### EXAMPLES

Plants Growth. All the experiments were carried out on 3-4 weeks old lab-grown plants. Seeds were purchased from David's gardens seeds (TX, USA) and Renee's Garden (CA, USA). Spinach (Spinacia oleracea, carmel and catalina), arugula (Eruca sativa), watercress (Nasturtium officinale) and kale (Brassica oleracea) were grown in a plant growth chamber (Adaptis 1000, Conviron, Canada) at set condition of 60% humidity, 18° C., medium light intensity, and 16 h light/8 h dark. The plant age was counted from seeding.

Preparation of dye conjugated silica nanoparticles (SNP-AF). Twenty-five microliters of (3-glycidyloxypropyl)trimethoxysilane (GPTS, Sigma, MO, USA) was added to 100 μL of 75% ethanol/water to be hydrolyzed for 1 h at room temperature. When GPTS was added to 0.5 mL of silica nanoparticles (10 mg/ml, Nanocomposix, CA, USA) in 2.5 mL of 80% of ethanol/water, then the temperature gradually increased up to 65° C. and the reaction was continued for 24 h. GPTS-silica nanoparticles were washed with ethanol and water multiple times by using centrifugal filter (Mw cut-off 30 kDa, Millipore, MA, USA) at 1,250 rpm for 15 min. Two hundred micrograms of Alexa Fluor 488-cadaverine (Invitrogen, MA, USA) was added to 2 mL of GPTS-SNP (1 mg/mL). This reaction was continued for another 24 h at 65° C. Alexa Fluor 488 conjugated silica nanoparticles (SNP-AF) was washed with water thoroughly until the filtrated solution had no detectable absorbance at 493 nm.

Preparation of BODIPY®FL encapsulated PLGA nanoparticles (PLGA-Bodipy). BODIPY® FL (Invitrogen), hydrophobic fluorescent dye, encapsulated PLGA nanoparticles were prepared by nanoprecipitation technique. See, Murakami, H., Kobayashi, M., Takeuchi, H. & Kawashima, Y. Preparation of poly(DL-lactide-co-glycolide) nanoparticles by modified spontaneous emulsification solvent diffusion method. Int J Pharm 187, 143-152 (1999), and Makadia, H. K. & Siegel, S. J. Poly Lactic-co-Glycolic Acid (PLGA) as Biodegradable Controlled Drug Delivery Carrier. Polymers (Basel) 3, 1377-1397, doi:10.3390/polym3031377 (2011), each of which is incorporated by reference in its entirety. One milligram of the dye was dissolved in 0.2 mL of acetone (Sigma), and 10 mg of PLGA (lactide:glycolide 50:50, Mw 30,000-60,000, Sigma) was dissolved in 0.3 mL of acetone. These are mixed together, and added to 2 mL of 1.5 wt % polyvinyl alcohol (PVA, Mw 31,000-50,000, Sigma) aqueous solution with vigorous stirring. The reaction was continued for 1 h followed by evaporation of acetone. The remained BODIPY® FL in the solution was removed by centrifugation at 8,000 rpm for 10 min.

Preparation of luciferase immobilized silica nanoparticles (SNP-Luc). Ten milliliters of GPTS-silica nanoparticles (10 mg/mL in water) was reacted with 200 mg of poly(ethylene glycol) bis(amine) (NH₂-PEG-NH₂, Mw 2,000, Sigma) for 6 h at 65° C. The excess PEG was removed thoroughly by using centrifugal filter (Mw cut-off 30 kDa) washing with water multiple times. The resulting SNP-PEG amine is kept at 4° C. until luciferase immobilization. Firefly luciferase (Promega, MI, USA) is physically anchored on PEG chain, and also has electrostatic interaction with positive charge of amine end groups.²²⁻²⁴ One milligram of luciferase (12.4 mg/mL) was incubated with 2 mg of SNP-PEG in 30 mM HEPES buffer (pH 7.4) for more than 1 h at 4° C. Unbound luciferase was gently removed by centrifugal filter tube (Mw cut-off 100 kDa) at 4° C.

Preparation of luciferin encapsulated PLGA nanoparticles (PLGA-LH). Luciferin encapsulated PLGA nanoparticles were prepared by aforementioned nanoprecipitation technique. Five milligram of D-luciferin (Sigma) was dissolved in 1 mL of acetone, and 50 mg of PLGA was dissolved in 1 mL of acetone. These are mixed together, and added to 8 mL of 1.5 wt % polyvinyl aqueous solution with vigorous stirring. The reaction was continued for 1 h followed by evaporation of acetone. The remaining luciferin in the solution was removed by centrifugal filter (Mw cut-off 100 kDa). As measured UV absorbance (λₘₐₓ=328 nm) of supernatant after centrifugation, encapsulation yield of luciferin were determined by 27%.

Preparation of coenzyme A functionalized chitosan-tripolyphosphate nanoparticles (CS-CoA). See, Venkatesan, C., Vimal, S. & Hameed, A. S. Synthesis and characterization of chitosan tripolyphosphate nanoparticles and its encapsulation efficiency containing Russell's viper snake venom. J Biochem Mol Toxicol 27, 406-411, doi:10.1002/jbt.21502 (2013), which is incorporated by reference in its entirety. Five milligrams of coenzyme A was mixed with 2 mg/mL of chitosan (medium Mw, Sigma) in 0.3% acetic acid. This mixture was slowly added dropwise to 2 mL of tripolyphosphate (TPP, Sigma) aqueous solution (1 mg/mL) with magnetic stirring. The reaction was continued for 2-3 h and the remained coenzyme A was removed by centrifugation at 8,000 rpm for 10 min. As measured UV absorbance (λₘₐₓ=258 nm) of supernatant after centrifugation, encapsulation yield of coenzyme A were determined by 39%.

Preparation of luciferase immobilized quantum dots (QD-Luc). Quantum dots functionalized with amine-derivatized PEG (λₑₘ=˜800 nm, 80 μM, Invitrogen) was conjugated with 1.5 equiv. of maleimide-PEG₂-succimidyl ester (Aldrich) for 1 h in 0.1 M-pH 7 phosphate buffer. The unbound maleimide-PEG₂-succimidyl ester was thoroughly removed by centrifugation (Mw cut-off 50 kDa) multiple times. Luciferase was covalently linked to the maleimide functional group of quantum dots in 0.1 M-pH phosphate buffer for 2 h at 4° C. The unbound luciferase was gently removed by centrifugal filter (Mw cut-off 100 kDa).

Dynamic light scattering and Zeta potential measurement. Dynamic Light Scattering (DLS) and Phase Analysis Light Scattering Zeta Potential Analyzer (PALS) were used to characterize nanoparticle surface charge and size distribution (NanoBrook ZetaPALS Potential Analyzer, NY, USA). The average particle size was determined using DLS (averaged over 3 runs) and the nanoparticle surface charge was determined using PALS zeta potential measurement, averaged over 10 runs.

Fourier transform infrared spectroscopy (FT-IR) spectroscopy. Characteristic peaks of functional groups introduced on silica nanoparticles were confirmed by FT-IR spectroscopy (Thermo Electron Co. WI, USA).

Water drop contact angle measurement. Contact angle of water drop on leaf surface of both leaf abaxial and adaxial side was measured (Model 200 with manually tilting base and Drop Image Advanced Software, Ramé-Hart, NJ, USA). A leaf was separated from a 3-4 weeks old plant and cut into an approximately 1×1 cm piece and was held in place using glass coverslips at each edge of the leaf. At least two independent measurements were carried out on both leaf adaxial and abaxial sides of each kind of leaf, and the contact angles were average of 10 measurements.

Spectrofluorometer measurement. Spectrofluorometer (Fluorolog-3, Horiba Jobin Yvon, Japan) was used to measure luminescence in vitro. The reaction mixture is total 750 including 30 mM pH 7.4 HEPES-MgCl₂ buffer, SNP-Luc, luciferin, ATP and optional coenzyme A and QD-Luc. The cuvette was placed in the spectrofluorometer, and the light emission was monitored under constant mixing with a magnetic stirrer (CIMARECi, Thermo Fisher Scientific, MA, USA). Leaf light emission was directly measured by inserting the leaf was in a sample holder.

Fluorescent confocal micrographs. Confocal images were taken in a Zeiss LSM 710 NLO microscope (Germany). HEPES-MgCl₂ buffer (30 mM, pH 7.4) alone or 0.15 mg/mL of SNP-AF in buffer was infiltrated into leaves as attached in the living plants by LIN or PBIN method. The leaf was cut immediately or in 2 h after infiltration, and leaf disc (5 mm in diameter) was prepared. Before submerge the leaf disc in FM-464 solution (Sigma, 10 μg/mL) to stain cell membranes, 5-10 holes was made in the lower side of the leaf to improve penetration of the dye. After another 2 h, the leaf disc was transferred to a glass slide having a polydimethylsiloxane (PDMS, Carolina Observation Gel, NC, USA) chamber filled with perfluorodecalin (PFD, Sigma) on glass slide. See, Littlejohn, G. R. & Love, J. A simple method for imaging Arabidopsis leaves using perfluorodecalin as an infiltrative imaging medium. J Vis Exp, doi:10.3791/3394 (2012), which is incorporated by reference in its entirety. The slide was sealed with a coverslip and image with a ×40 water immersion objective.

Infiltration of nanoparticles in living plants. Leaf laminar infiltration of nanoparticles (LIN) technique requires nanoparticle suspension to be infiltrated through the leaf abaxial side of leaf using a 1 mL-volume syringe (NORM-JECT, Germany). For pressurized bath infusion of nanoparticles (PBIN), a whole plant is submerged inside a 100 mL-volume glass body syringe (Hamilton, NV, USA) with luer lock valve containing the nanoparticle suspension and followed by pressurization using a syringe pump (KD Scientific Inc. MA, USA). The pressure was monitored by digital hydronic manometer (Dwyer instruments, IN, USA). After infusion of nanoparticles with LIN or PBIN, the infiltrated plants were thoroughly washed with water to remove the remaining nanoparticles on the surfaces. The nanoparticle suspension for PBIN was prepared in 80 mL of 30 mM-pH 7.4 HEPES-MgCl₂ buffer including PLGA-luciferin nanoparticles (luciferin 140 μM) and optional CS-CoA nanoparticles (coenzyme A 33 μM).

Fabrication of syringe applicator. The syringe applicators were designed in AutoCad, and fabricated by using a LulzBot Mini Desktop 3D printer (Aleph Objects, Inc. CO, USA) with plastic filament (High impact polystyrene; HIPS, 3 mm).

Estimation of photon numbers from light emitting plants. The power of LED light was first measured at 12.70 cm away using a photo-detector (λ=530 nm, detector surface area=1 cm²) (PM100, ThorLabs, NJ, USA). The number of photons incident on the photo-detector was determined using the measured power (10 nW) divided by the energy of a single photon at 530 nm, given by the equation below

$${E}_{photon} = hf = \frac{hc}{λ} = \frac{( 6.63 E - 34 ) ( 3 E 8 )}{530 E - 9} = 0.0375 E - 17 J$$

where Eₚₕₒₜₒₙ is the energy of 1 photon at 530 nm, h is the Plancks constant, c and λ are the speed of light (3×10⁸ m/s), and the wavelength (530 nm), respectively. The total photons/s from the LED light (assuming point source) is

$$\frac{4 π {r}^{2}}{1} ( 10 nW ) = 20270 nW = 5.47 E 13 photons \text{/} s$$

where r is the distance from the source.

An image of the LED light was taken at 68.58 cm of distance with 1/1000 s exposure to avoid saturation of pixels and converted into quantitative values using ImageJ (Mean value=0.016). This corresponded to an incident 1.44E5 photons/s on the camera aperture (0.155 cm², f=20 mm). An image of the light emitting plant was similarly taken at 24.13 cm with 30 s exposure and converted into quantitative values using ImageJ (Mean value=4.6), corresponding to an incident 1.42E6 photons/s on the camera aperture (0.349 cm², f=30 mm). The total photons/s from the light emitting plant can hence be estimated to be 2.98E10 photons/s as assuming 1/r² intensity dependence.

Detection of nIR emission with Raspberry Pi. A Raspberry Pi® equipped with a f=3.6 mm 1/2.7″ camera with IR filters removed (SainSmart Infrared Night Vision Surveillance Camera, KS, USA) was used. To detect nIR emission from the QD-Luc embedded within the living plant, a FEL0750 long pass filter (ThorLabs Inc.) was placed in front of the camera lens, and images were collected at 6 s exposure with ISO 800. The mixture infiltrated into the watercress plant comprised of 100 μL 30 mM pH 7.4 HEPES-MgCl₂ buffer containing QD-Luc or SNP-Luc, 100 μM free luciferin, and with or without 100 μM ATP.

Characterization of Functionalized Silica Nanoparticles by FT-IR Spectroscopy

| Nanoparticles | FT-IR wavenumbers (cm⁻¹) | |
|---|---|---|
| SNP | 972.1 | (Si—OH) |
| SNP-GPTS | 2863.9, 2926.9 | (—CH₂—) |
| SNP-PEG amine | 3310.8 | (O—H) |
| | 2920.9 | (—CH₂—) |
| | 1457.1 | (—CH₂—) |
| | 1093.3 | (C—O) |
| | 925.7 | (C—O—C) |
| SNP-Luc | 3275.1 | (O—H) |
| | 2938.6, 2876.4 | (—CH₂—) |
| | 1456.4 | (—CH₂—) |
| | 1652 | (C═O) |
| | 1035.0 | (C—O) |
| | 923.2 | (C—O—C) |

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

1. A method of delivering a composition into a plant, comprising:
submerging the plant in an chamber, wherein the chamber contains water and the composition; and
applying an external pressure to the chamber, thereby generating an inward flow through stomata pores of a plant leaf and infiltrating the composition into the plant.

2. The method of claim 1, further comprising localizing the composition in an organelle, a cell, or a tissue of the plant.

3. The method of claim 2, wherein the organelle is selected from the group consisting of a nucleus, endoplasmic reticulum, Golgi apparatus, chloroplast, chromoplast, gerontoplast, leucoplast, lysosome, peroxisome, glyoxysome, endosome and vacuole.

4. The method of claim 2, wherein the cell is a stomata guard cell.

5. The method of claim 2, wherein the tissue is mesophyll.

6. The method of claim 1, wherein the external pressure is no less than 1.8 bar.

7. The method of claim 1, wherein a water contact angle on a surface of the plant is less than 113°.

8. The method of claim 1, wherein the external pressure is applied at a velocity less than 0.4 bar/s.

9. The method of claim 1 wherein the composition includes particles having a size of less than 20 nm.

10. The method of claim 1, wherein the composition includes particles having a size of less than 10 nm.

11. The method of claim 1, wherein the composition includes a nanoparticle.

12. The method of claim 11 wherein a light emitting compound is immobilized on the nanoparticle.

13. The method of claim 12, wherein the light emitting compound is luciferase.

14. The method of claim 11, wherein the nanoparticle includes a nanotube, a carbon nanotube, or a single-walled carbon nanotube.

15.-16. (canceled)

17. The method of claim 11, wherein the nanoparticle includes a polymer.

18. The method of claim 17, wherein the polymer includes a polynucleotide.

19. The method of claim 18, wherein the polynucleotide includes poly(AT).

20. The method of claim 17, wherein the polymer includes a polysaccharide.

21. The method of claim 20, wherein the polysaccharide is selected from the group consisting of dextran, pectin, hyaluronic acid, chitosan, and hydroxyethylcellulose.

22. The method of claim 17, wherein the polymer includes poly(ethylene glycol).

23. The method of claim 11, wherein the nanoparticle is photoluminescent.

24. The method of claim 11, wherein the nanoparticle emits near-infrared radiation.

25. The method of claim 11, wherein the nanoparticle is photoluminescent and the photoluminescence emission of the photoluminescent nanoparticle is altered by a change in a stimulus within the plant.

26. The method of claim 25, wherein the stimulus is a concentration of an analyte.

27. The method of claim 26, wherein the analyte is a reactive oxygen species, nitric oxide, carbon dioxide, adenosine triphosphate, nicotinamide adenine dinucleotide phosphate, oxygen, or methane.

28.-33. (canceled)

34. The method of claim 25, wherein the stimulus is a pH of an organelle of the plant.

35. The method of claim 11, wherein the nanoparticle is a semiconductor.

36. The method of claim 1, wherein the composition includes a dye, an enzyme, a nutrient, or a gene.

37.-39. (canceled)

40. A green plant comprising a composition including:
a nanoparticle,
a silane conjugated with the nanoparticle; and
a dye conjugated with the nanoparticle.

41. The green plant of claim 40, wherein the silane is (3-glycidyloxypropyl)trimethoxysilane.

42. The green plant of claim 40, wherein the nanoparticles includes silica.

43. A green plant comprising a composition including:
a nanoparticle,
a polymer conjugated with the nanoparticle; and
a light-emitting compound immobilized on the nanoparticle via the polymer or an enzyme immobilized on the nanoparticle via the polymer.

44. The green plant of claim 43, wherein the polymer includes poly(ethylene glycol).

45. The green plant of claim 43, wherein the light-emitting compound or enzyme is luciferase.

46. A green plant comprising a composition including a nanoparticle encapsulated by a light-emitting compound.

47. The green plant composition of claim 46, wherein the light-emitting compound is luciferin.

48. A green plant comprising a composition including:
a plurality of nanoparticles; and
a polysaccharide conjugated with each of the plurality of nanoparticles, wherein a chemical compound is encapsulated by the plurality of nanoparticles.

49. The green plant of claim 48, wherein the polysaccharide is chitosan.

50. The green plant of claim 48, wherein the chemical compound is coenzyme A.

51.-53. (canceled)
